(19) United States
(12) Patent Application Publication
(10) Pub. No.: US 2016/0206189 A1
(43) Pub. Date: Jul. 21, 2016

(54) FACILITATING TRACHEAL INTUBATION USING AN ARTICULATING AIRWAY MANAGEMENT APPARATUS

(71) Applicants: University Hospitals of Cleveland, Cleveland, OH (US); Amin, Turocy & Watson, LLP, Cleveland, OH (US)

(72) Inventors: Howard S. Nearman, Pepper Pike, OH (US); Donald M. Voltz, Twinsburg, OH (US); Alon S. Aharon, Scarsdale, NY (US)

(21) Appl. No.: 15/084,028

(22) Filed: Mar. 29, 2016

Related U.S. Application Data
(63) Continuation-in-part of application No. 13/914,472, filed on Jun. 10, 2013, now Pat. No. 9,326,669.
Application No. 13/914,472 is a division of application No. 12/636,271, filed on Dec. 11, 2009, now Pat. No. 8,460,184.

(51) Int. Cl. A61B 1/267; A61B 5/06; A61B 1/008; A61B 1/00; A61B 1/04; A61B 5/083

(52) U.S. Cl. CPC A61B 1/267; A61B 1/04; A61B 5/0836; A61B 1/008; A61B 1/00016; A61B 1/00006; A61B 5/065

(57) ABSTRACT

The claimed subject matter provides systems and/or methods that facilitate improving intubating a patient. An airway management apparatus is provided having an articulating blade comprising a plurality of articulation points and a channel configured to receive an endotracheal tube and facilitate passage of the endotracheal tube through the channel. The airway management apparatus further include a handle attached to the articulating blade and comprising a housing, a motor provided within the housing that is operatively coupled to the articulating blade, and a blade control component. The blade control component is provided on or within the housing, wherein the blade control component is operatively coupled to the motor and controls manipulation of the articulating blade at the plurality of articulation points via the motor.

### CROSS-REFERENCE TO RELATED APPLICATIONS

This application is a continuation-in-part of U.S. patent application Ser. No. 13/914,742 filed on Jun. 10, 2013, and entitled “AIRWAY MANAGEMENT,” which is a divisional of U.S. patent application Ser. No. 12/636,271, filed Dec. 11, 2009, and entitled “AIRWAY MANAGEMENT,” which is a U.S. national stage filing of Patent Cooperation Treaty (PCT) application serial number PCT/US08/66544 filed Jun. 11, 2008, entitled “AIRWAY MANAGEMENT,” which claims the benefit of U.S. Provisional Patent Application Ser. No. 60/943,320 filed Jun. 12, 2007, entitled “AIRWAY MANAGEMENT.” The entireties of the aforementioned applications are herein incorporated by reference.

### TECHNICAL FIELD

This disclosure generally relates to facilitating tracheal intubation using an articulating airway management apparatus.

### BACKGROUND

Medical endoscopy has continued to advance with increasing sophistication in both camera and illumination technology. The area of airway management has also embraced technological advances in optics and light transmission resulting in development of numerous devices to assist a medical provider with placement of a breathing endotracheal tube into the trachea of a patient requiring mechanical ventilatory assistance (e.g., endotracheal intubation).

An area of airway management which has not seen much advancement since the introduction of peroral endotracheal intubation in the 18th century is the design of the laryngoscopic instrument used to displace the tongue and allow for visualization of vocal cords and laryngeal aperture. A number of subtle changes have been implemented in these tools resulting in many different variations in the laryngoscopic articulating blade. These devices, although quite varied in design, are placed into the oral cavity and used to forcefully move the tongue, mandible, and connected soft tissue out of the way allowing for visualization of the tracheal inlet. This maneuver can be highly stimulating to patients necessitating some form of anesthesia to tolerate its use. In addition, even with increasing levels of force applied to the device, there are patients with anatomical variants or pathologic conditions that do not allow direct visualization of the tracheal opening.

In the United States, it has been estimated that 10 million people undergo general anesthesia each year for a variety of operations. During the induction of general anesthesia, a significant percentage of patients require placement of an endotracheal tube along with mechanical ventilation to overcome cessation of breathing caused by anesthetic medications. The process of placing an endotracheal tube into the trachea varies in difficulty depending on a patient's body habitus, variations in normal anatomy, as well as variations in anatomic deviations as a result of numerous pathologic processes. Placement of the endotracheal tube depends both on the skills of the anesthesiologist as well as the instruments used to visualize the opening of the trachea. In a normal anesthetic situation, once a patient is placed under general anesthesia, a rigid laryngoscope can be placed into the mouth to displace the tongue allowing for exposure of the laryngeal aperture. Once the larynx is visualized, an endotracheal tube can be placed into the trachea and a high volume, low pressure cuff can be inflated to provide a seal between the endotracheal tube and the inner wall of the trachea.

Numerous risks and complications can occur with the placement of an endotracheal tube, risks that increase in patients with abnormal body habitus (such as morbid obesity), or variations in normal anatomy as the result of congenital or pathologic conditions. Thus, anesthesiologists desire to quickly, reliably and safely place an endotracheal tube after anesthetic induction to mitigate chances of the patient becoming hypoxic (e.g., lack of oxygen in the blood) resulting in injury to systems in the body, especially the heart and the brain. For example, it has been estimated that intubation problems account for about one third of all deaths and serious injuries related to anesthesiology. In addition, many more patients are placed at risk outside the operating room. For instance, emergent placement of an endotracheal tube can be encountered when a patient experiences cardiac and/or respiratory arrest, both inside and outside the hospital setting. A challenge for anesthesiologists as well as other health care providers who have specialty training in the area of airway management is to place the endotracheal tube in a position far removed from where they are visualizing it (e.g., viewing from the mouth opening for traditional laryngoscopy).

### SUMMARY

The following presents a simplified summary in order to provide a basic understanding of some aspects described herein. This summary is not an extensive overview of the claimed subject matter. It is intended to neither identify key or critical elements of the claimed subject matter nor delineate the scope thereof. Its sole purpose is to present some concepts in a simplified form as a prelude to the more detailed description that is presented later. The claimed subject matter provides systems and/or methods that facilitate improving intubating a patient.

In one or more embodiments, an airway management apparatus is provided having an articulating blade comprising a plurality of articulation points and a channel configured to receive an endotracheal tube and facilitate passage of the endotracheal tube through the channel. The airway management apparatus further include a handle attached to the articulating blade and comprising a housing, a motor provided within the housing that is operatively coupled to the articulating blade, and a blade control component. The blade control component is provided on or within the housing, wherein the blade control component is operatively coupled to the motor and controls manipulation of the articulating blade at the plurality of articulation points via the motor. In some implementations, the airway management apparatus can also include a tube control component provided on or within the housing. The tube control component is operatively coupled to the motor or another motor provided within the housing that is configured to operatively couple to the endotracheal tube when the endotracheal tube is provided within the channel. The tube control component controls passage of the endotracheal tube through the channel via the motor or the other motor.

In other implementations, the airway management apparatus of claim further includes one or more sensor devices located on or within the articulating blade that are configured to capture sensor data during usage of the airway management apparatus in association with performance of a tracheal intubation procedure, including at least one of: a camera, a chemical sensor, or a pressure sensor. In some aspects of this implementation, the airway management apparatus can include a memory that stores computer executable components, and a processor that executes at least the computer executable components stored in the memory, including a targeting component configured to determine, based on the sensor data, a location of the articulating blade relative to a target location within an oral cavity of a patient during the performance of the intubation procedure, and further determine a movement for the articulating blade based on the location.

In various additional embodiments, an airway management apparatus is provided that includes an articulating blade having a plurality of articulation points, a handle having a housing that is attached to the articulating blade, and a motor provided within the housing that is operatively coupled to the articulating blade. The airway management apparatus further includes a blade control component, provided on or within the housing, that is operatively coupled to the motor and controls manipulation of the articulating blade at the plurality of articulation points via the motor, and one or more sensor devices located on or within the articulating blade that are configured to capture sensor data during usage of the airway management apparatus in association with performance of a tracheal intubation procedure, wherein the one or more sensor devices comprise at least one of a: a camera, a chemical sensor, or a pressure sensor.

In one or more additional embodiments, the airway management apparatus further includes a memory that stores computer executable components, and a processor that executes computer executable components stored in the memory. These computer executable components can include an analysis component configured to determine a movement for the articulating blade based on the sensor data. In one implementation, the computer executable components further include a guidance component configured to generate an output that informs an operator of the airway management apparatus regarding the movement. In another implementation, the analysis component is further configured to direct the blade control component to cause the articulating blade to perform the movement.

The subject disclosure further provides various methods for facilitating performance of a tracheal intubation procedure using an airway management apparatus. In one embodiments, a method includes receiving, by a device including a processor, sensor data from one or more sensor devices located on or within an articulating blade of the airway management apparatus during insertion of the articulating blade into an oral cavity of a patient in association with performance of the tracheal intubation procedure on the patient, wherein the one or more sensor devices comprise at least one of: a camera, a chemical sensor, or a pressure sensor. The method further includes, determining, by a the device, a movement for the articulating blade within the oral cavity based on the sensor data, including determining a manner of articulation for the articulating blade via an articulation joint of the articulating blade.

In one implementation, the device is a remote device located remote from the airway management apparatus. According to this implementation, the method can further include generating, by the device, an output that informs an operator of the airway management apparatus regarding the movement. The method can also include sending, by the device to the airway management apparatus, a command with information instructing the airway management apparatus to perform the movement, wherein the articulating blade is configured to automatically articulate according to the movement based on reception of the command.

In another implementation, the device is a handle of the airway management apparatus that is physically attached to the articulating blade. According to this implementation, the method can further include, generating, by the device, an output signal that informs an operator of the airway management apparatus regarding the movement. The method can also include, controlling, by the device, a motor of the airway management apparatus operatively coupled to the articulating blade, to cause the articulating blade to articulate according to the movement based on the determining. In yet another aspect, the method can include determining, by the device, a location of the articulating blade relative to a target location within the oral cavity based on the sensor data. For example, the sensor data can include the sensor data one or more images of internal anatomical features located within the patient's oral cavity and an end-tidal carbon dioxide (EtCO₂) level. According to this aspect, the movement for the articulating blade is determined based on the location to facilitate brining the articulating blade to the target location.

The following description and the annexed drawings set forth in detail certain illustrative aspects of the claimed subject matter. These aspects are indicative, however, of but a few of the various ways in which the principles of such matter may be employed and the claimed subject matter is intended to include all such aspects and their equivalents. Other advantages and novel features will become apparent from the following detailed description when considered in conjunction with the drawings.

### BRIEF DESCRIPTION OF THE DRAWINGS

FIG. 1 illustrates an example system that facilitates tracheal intubation using an articulating airway management apparatus in accordance with various embodiments of the subject disclosure.

FIG. 2 presents an external view of a conventional tracheal intubation procedure in accordance with aspects and embodiment of the subject disclosure.

FIGS. 3 and 4 depict the vocal cords and the laryngeal aperture of a human patient.

FIG. 5 illustrates an example endotracheal tube that can be utilized in connection with airway management apparatuses described herein.

FIG. 6 illustrates bag-mask ventilation in accordance with conventional tracheal intubation procedures.

FIG. 7 presents another example airway management apparatus in accordance with one or more embodiments described herein.

FIGS. 8 and 9 present various features of an airway management including a channel configured to receive an endotracheal tube in accordance with various aspects and embodiments described herein.

FIGS. 10 and 11 present various features of an airway management including a channel having an endotracheal tube provided therein in accordance with various aspects and embodiments described herein.

FIGS. 12 and 13 present an enlarged perspective of a distal end of an airway management apparatus including a channel having an endotracheal tube provided therein in accordance with various aspects and embodiments described herein.

FIGS. 14-19 present various perspectives of an airway management apparatus configured to guide an endotracheal tube into a patient's trachea with the retainer segments removed in accordance with one or more embodiments described herein.

FIG. 20 presents a high-level block diagram of an example airway management apparatus in accordance with one or more embodiments described herein.

FIG. 21 presents a high-level block diagram of another example airway management apparatus in accordance with one or more embodiments described herein.

FIGS. 22-25 provide a pictorial demonstration of an example intubation procedure facilitated using an airway management apparatus including one or more sensors configured to facilitate guiding the airway management apparatus to a target location, in accordance with various aspects and embodiments described herein.

FIG. 26 illustrates another example system that facilitates tracheal intubation using an articulating airway management apparatus in accordance with various embodiments of the subject disclosure.

FIG. 27 presents a high-level block diagram of another example airway management apparatus in accordance with one or more embodiments described herein.

FIG. 28 presents a high-level block diagram of an example remote airway management device in accordance with one or more embodiments described herein.

FIGS. 29-34 provide example methods facilitates intubating a patient using an airway management apparatus in accordance with one or more aspects and embodiments described herein.

FIG. 35 illustrates an exemplary networking environment, wherein the novel aspects of the claimed subject matter can be employed.

FIG. 36 illustrates an exemplary operating environment that can be employed in accordance with the claimed subject matter.

### DETAILED DESCRIPTION

The claimed subject matter is described with reference to the drawings, wherein like reference numerals are used to refer to like elements throughout. In the following description, for purposes of explanation, numerous specific details are set forth in order to provide a thorough understanding of the subject innovation. It may be evident, however, that the claimed subject matter may be practiced without these specific details. In other instances, well-known structures and devices are shown in block diagram form in order to facilitate describing the subject innovation.

As utilized herein, terms “component,” “system,” and the like are intended to refer to a computer-related entity, either hardware, software (e.g., in execution), and/or firmware. For example, a component can be a process running on a processor, a processor, an object, an executable, a program, and/or a computer. By way of illustration, both an application running on a server and the server can be a component. One or more components can reside within a process and a component can be localized on one computer and/or distributed between two or more computers.

Additionally, the following description refers to components being “connected” and/or “coupled” to one another. As used herein, unless expressly stated otherwise, the terms “connected” and/or “coupled” mean that one component is directly or indirectly connected to another component, mechanically, electrically, wirelessly, inductively or otherwise. Thus, although the figures can depict example arrangements of components, additional and/or intervening components can be present in one or more embodiments.

Furthermore, the claimed subject matter may be implemented as a method, apparatus, or article of manufacture using standard programming and/or engineering techniques to produce software, firmware, hardware, or any combination thereof to control a computer to implement the disclosed subject matter. The term “article of manufacture” as used herein is intended to encompass a computer program accessible from any computer-readable device, carrier, or media. For example, computer readable media can include but are not limited to magnetic storage devices (e.g., hard disk, floppy disk, magnetic strips, . . . ), optical disks (e.g., compact disk (CD), digital versatile disk (DVD), . . . ), smart cards, and flash memory devices (e.g., card, stick, key drive, . . . ). Additionally it should be appreciated that a carrier wave can be employed to carry computer-readable electronic data such as those used in transmitting and receiving electronic mail or in accessing a network such as the Internet or a local area network (LAN). Of course, those skilled in the art will recognize many modifications may be made to this configuration without departing from the scope or spirit of the claimed subject matter. Moreover, the word “exemplary” is used herein to mean serving as an example, instance, or illustration. Any aspect or design described herein as “exemplary” is not necessarily to be construed as preferred or advantageous over other aspects or designs.

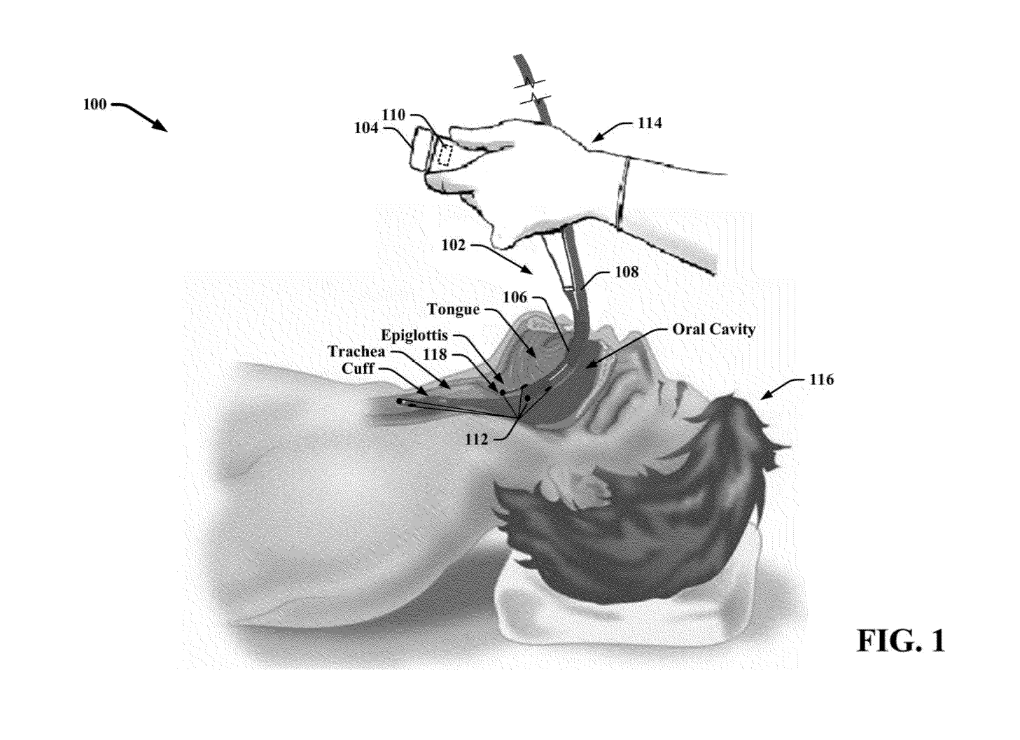

Referring now to FIG. 1, presented is an example system 100 that facilitates tracheal intubation in accordance with various aspects and embodiments described herein. System 100 includes a patient 116, an airway management apparatus 102 and an operator 114 or the airway management apparatus 102. The operator 114 can include for example, a physician (e.g., traditionally an anesthesiologist), a medical technician, a nurse, or other suitable trained medical professional. The airway management apparatus 102 is configured to facilitate placement of an endotracheal tube 108 into the trachea of the patient 116 while minimizing complications associated with conventional tracheal intubation.

The process of placing an endotracheal tube 108 into the trachea varies in difficulty depending on a patient's body habitus, variations in normal anatomy, as well as variations in anatomic deviations as a result of numerous pathologic processes. Placement of the endotracheal tube 108 depends both on the skills of the medical professional performing the intubation procedure as well as the instruments used to visualize the opening of the trachea.

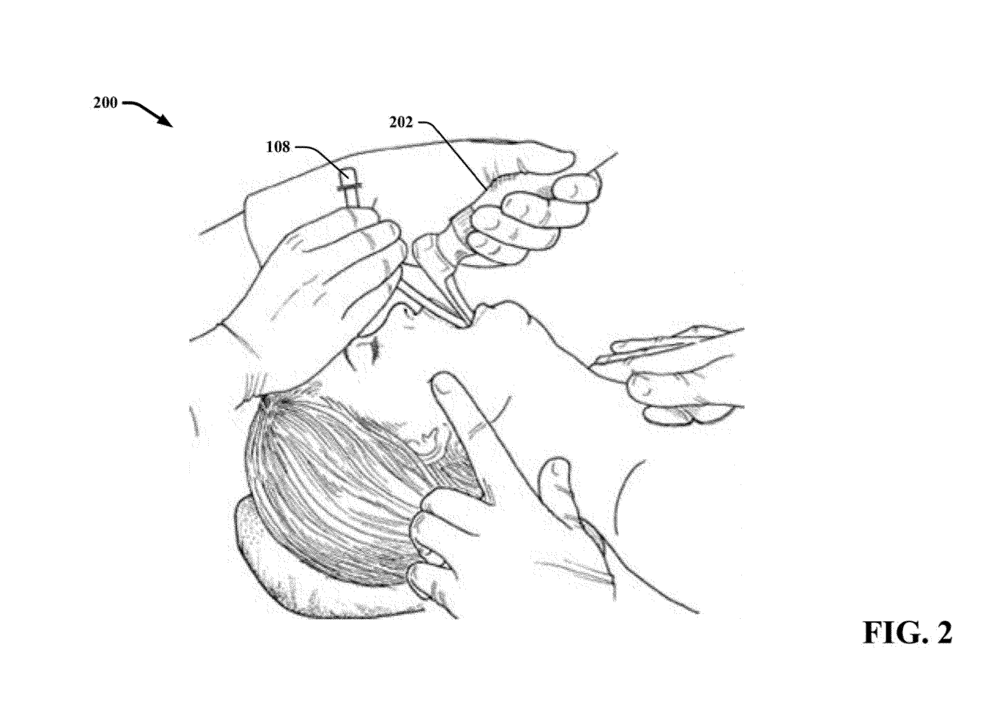

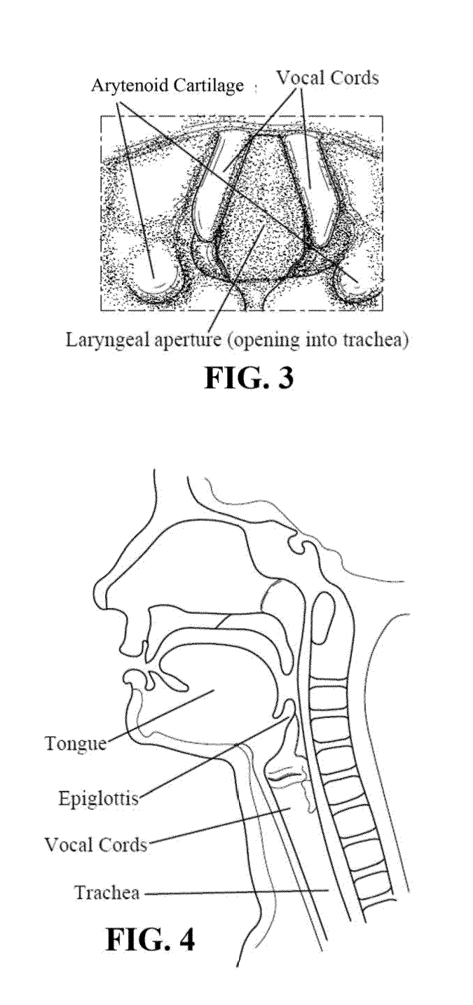

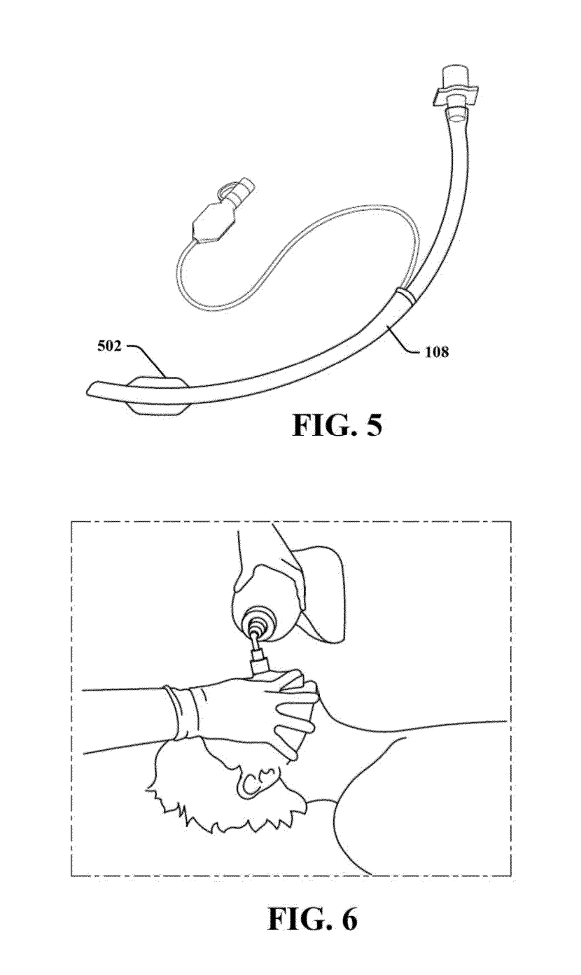

FIG. 2 presents an external view of a conventional tracheal intubation procedure 200. FIGS. 3 and 4 depict the vocal cords and laryngeal aperture. FIG. 5 illustrates an example endotracheal tube 108 that can be utilized in connection with the airway management apparatuses described herein. FIG. 6 illustrates bag-mask ventilation.

With reference to FIGS. 2-6, in a conventional anesthetic situation, once a patient is placed under general anesthesia, a rigid laryngoscope 202 can be placed into the mouth to displace the tongue allowing for exposure of the laryngeal aperture. Once the larynx is visualized, an endotracheal tube can be placed into the trachea and a high volume, low pressure cuff 502 can be inflated to provide a seal between the endotracheal tube and the inner wall of the trachea. Numerous risks and complications can occur with the placement of an endotracheal tube 108. These risks increase in patients with abnormal body habitus (such as morbid obesity), or variations in normal anatomy as the result of congenital or pathologic conditions. Thus, anesthesiologists desire to quickly, reliably and safely place an endotracheal tube 108 after anesthetic induction to mitigate chances of the patient becoming hypoxic (e.g., lack of oxygen in the blood) resulting in injury to systems in the body, especially the heart and the brain.

A typical example operating room intubation scenario proceeds as follows. A patient who is spontaneously breathing on their own is placed in a supine position and supplemental oxygen is provided in an attempt to “fill” their lungs, blood, and tissues with higher than normal oxygen levels (i.e., hyperoxygenation) to prevent a fall in oxygen levels and deaturation of the oxygen carrying molecules hemoglobin in the blood during the period when the patient is not breathing (e.g., as a result of the administration of anesthetic drugs that render patients unconscious and apneic (not breathing on their own)). Typically, during hyperoxygenation, an anesthesiologist has about 2-3 minutes to place the endotracheal tube 108 into the trachea before the patient becomes hypoxic requiring the addition of supplemental oxygen delivered with bag-mask ventilation (as shown in FIG. 6). Bag-mask ventilation is extremely difficult and in certain situation not possible. As a result, the patient may suffer severe hypoxia and potentially death or irreversible brain damage. Delays in securing an airway with the proper placement of an endotracheal tube 108 also extend the duration of anesthesia and add potential physiologic derangements that are poorly tolerated in certain patient populations, especially the elderly.

Complications with intubation do not end with those associated with an inability to properly place the endotracheal tube 108 within a short time frame (e.g., 2-3 minutes). Placement of a rigid laryngoscope 202 into someone's mouth to forcefully move the tongue, lower jaw, and upper airway soft tissue to reveal the laryngeal aperture is highly stimulating (e.g., of a cough or gag reflex) and not reliably blunted with standard anesthetic induction medications. Endotracheal intubation can result in severe physiologic stresses in patients that often lead to increases in heart rate and blood pressure in the adult population, and a precipitous fall in heart rate in pediatric patients. These stresses are not well tolerated in certain patient groups with co-existing heart conditions or those already at physiologic extremes (such as trauma patients). In addition, once anesthesia is administered to allow for intubation, the patient's airway can become compromised by a relaxation of the upper airway musculature resulting in an obstruction that can be very difficult to overcome with bag-mask ventilation or the use of other airway devices.

Furthermore, in certain airway situations, the placement of an endotracheal tube 108 can only safely be accomplished by keeping a patient in an awake and spontaneously breathing state. In these situations, it is of paramount importance that medical professional performing intubation is able to adequately anesthetize the upper airway to blunt the cough reflex as well as to blunt any painful stimuli these patients would experience with the placement of the intubation equipment.

Referring back to FIG. 1, the subject airway management apparatus 102 provides various advanced features and functionality that facilitate minimizing the aforementioned complications associated with conventional tracheal intubation. Airway management apparatus 102 decreases the time required for intubation, decreases the stress on the patient, and minimizes human errors in association with performance of the intubation procedure. Airway management apparatus 102 includes a handle 104 with an articulating laryngoscope blade 106 attached thereto. The articulating blade 106 is configured to articulate at one or more articulation points or joints of the articulating blade (not shown) to adapt to the curvature of a patient's oral anatomy, thereby facilitating intubation. For example, in one or more embodiments, the articulating blade 106 can be constructed of a plurality of flat metal articulating blades that articulate on one another allowing for the articulating blade 106 to dynamically assume multiple configurations depending on the patient's airway anatomy. Thus, the articulating blade 106 can include multiple articulating plates that allow the articulating blade 106 to flex throughout its length as well as at the distal end 118 of the articulating blade 106.

A mechanical and/or electrical control means is provided within the handle 104 that controls articulation and movement of the articulating blade 106 prior to and during the intubation procedure. For example, the handle 104 can include a housing having a shape that facilitates holding and operating the airway management apparatus 102 with a single hand. The housing can house various mechanical and electrical components and one or more power sources. The electrical components can be powered via the one or more power sources. A suitable power source can include, but is not limited to, a battery, a capacitor, a charge pump, a mechanically derived power source (e.g., microelectromechanical systems (MEMs) device), or an induction component. Additionally, the handle 104 can include an interface that enables connecting to a cradle. When connected to (e.g., docked upon) the cradle, the power source can be recharged.

The electrical components provided within the housing of the handle 104 can vary depending on the particular features and functionality of the airway management apparatus 102. In various embodiments, the electrical components include one or more motors (not shown) configured to manipulate the shape and configuration of the articulating blade 106, such as servo-motor, a linear motor, or another suitable motor. The electrical components can also include, but are not limited to, one or more processors, memories, transmitters, receivers, transceivers, sensors, sensing circuitry, therapy circuitry, antennas and other components. In an embodiment, the electrical components can be formed on or within a substrate that is placed inside the housing of the handle 104. The housing can be formed from conductive materials, non-conductive materials or a combination thereof. For example, housing can include a conductive material, such as metal or metal alloy, a non-conductive material such as glass, plastic, ceramic, etc., or a combination of conductive and non-conductive materials.

The handle 104 can also include controls that allow for manipulation of the articulating blade 106 via the one or more motors provided within the housing (e.g., a servo-motor, a linear motor, etc.). The controls can provide varying degrees of control precision (e.g., medium control, fine control, etc.) of the articulating blade 106. By way of illustration, the controls included with the handle 104 can mechanically alter the size, shape, curvature, orientation, etc. of the articulating blade 106. For example, in the embodiment shown, the handle 104 (e.g., within the housing of the handle) can include a control circuit 110 configured to control mechanical adjustment of the articulating blade 106 via the one or more motors using an electrical signal, and so forth. The control circuit 110 can be configured to receive input defining a manner of movement for the articulating blade 106, interpret the input, and direct, via an electrical signal, a motor physically coupled to the articulating blade 106 to cause the articulating blade 106 to perform the movement. For instance, the movement can include articulation via the one or more articulation points in substantially any direction, elongation of the articulating blade, shortening of the articulating blade, elevation of the articulating blade, etc., or a portion thereof.

In some embodiments, the airway management apparatus 102 is a self-contained single unit wherein the handle 104 and the articulating blade 106 are permanently physically attached. In other embodiments, the articulating blade 106 can be configured to attach and detach from the handle 104. According to these embodiments, articulating blades of different sizes, shapes, thicknesses, material compositions, functionality, etc., can be attached to the handle 104 interchangeably.

The articulating blade 106 facilitates depressing the patient's tongue, pulling back the epiglottis, and exposing the patient's trachea (e.g., via exposure of the laryngeal aperture) and vocal cords. After the patient's trachea and vocal cords are exposed, an endotracheal tube 108 can be inserted into the patient's trachea. In some embodiments, the endotracheal tube 108 is inserted into the patient's trachea in a freestyle manner, completely detached from the articulating blade 106. For example, similar to procedure 200 depicted in FIG. 2, the operator 114 of the airway management apparatus 102 can hold the airway management apparatus 102 with one hand while freely guiding the endotracheal tube 108 into the patient's trachea using the other hand. According to this embodiment, the airway management apparatus does not include the endotracheal tube 108 partially attached to the articulating blade 106 or otherwise integrated thereon.

In another embodiment, as shown in system 100, the articulating blade 106 of the airway management apparatus 102 is configured to guide the endotracheal tube 108 into the correct position within the patient's trachea. According to this embodiment, the endotracheal tube 108 is configured to partially attach to the articulating blade 106 such that the endotracheal tube 108 follows the curvature of the articulating blade 106. The endotracheal tube 108 thus moves with the contour of the articulating blade 106 as it articulates via the one or more articulation points or joints. The articulating blade 106 facilitates passage of the endotracheal tube 108 over the length of the articulating blade 106, past the distal end 118 of the articulating blade 106, and into the patient's trachea. After the endotracheal tube 108 is correctly positioned within the patient's trachea, the articulating blade 106 is detached from the endotracheal tube 108 and removed from the patient's oral cavity.

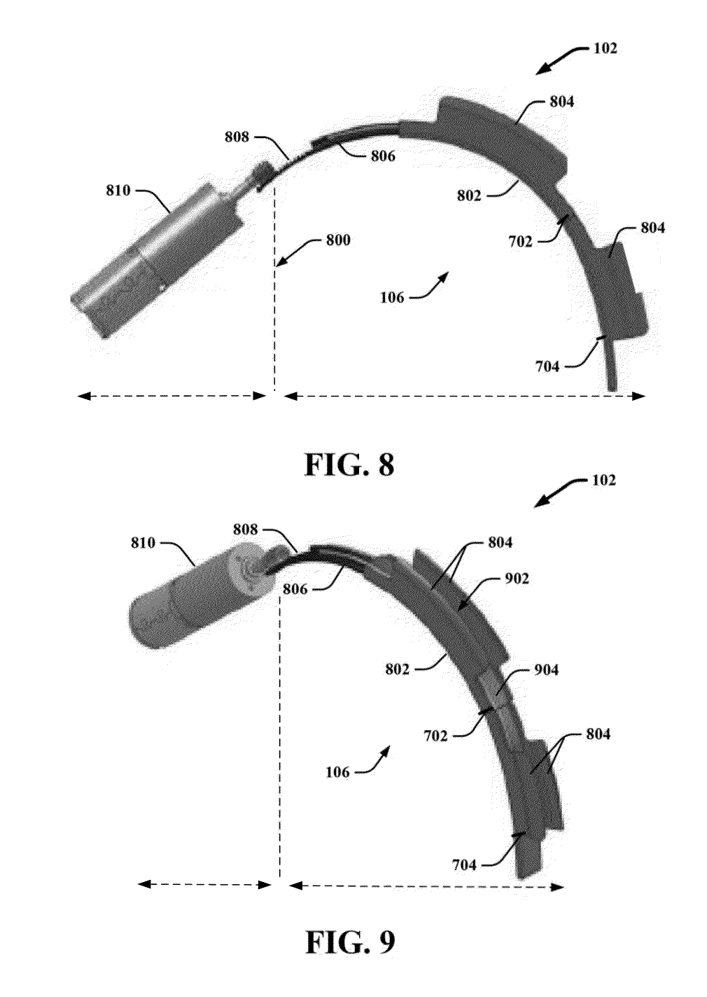

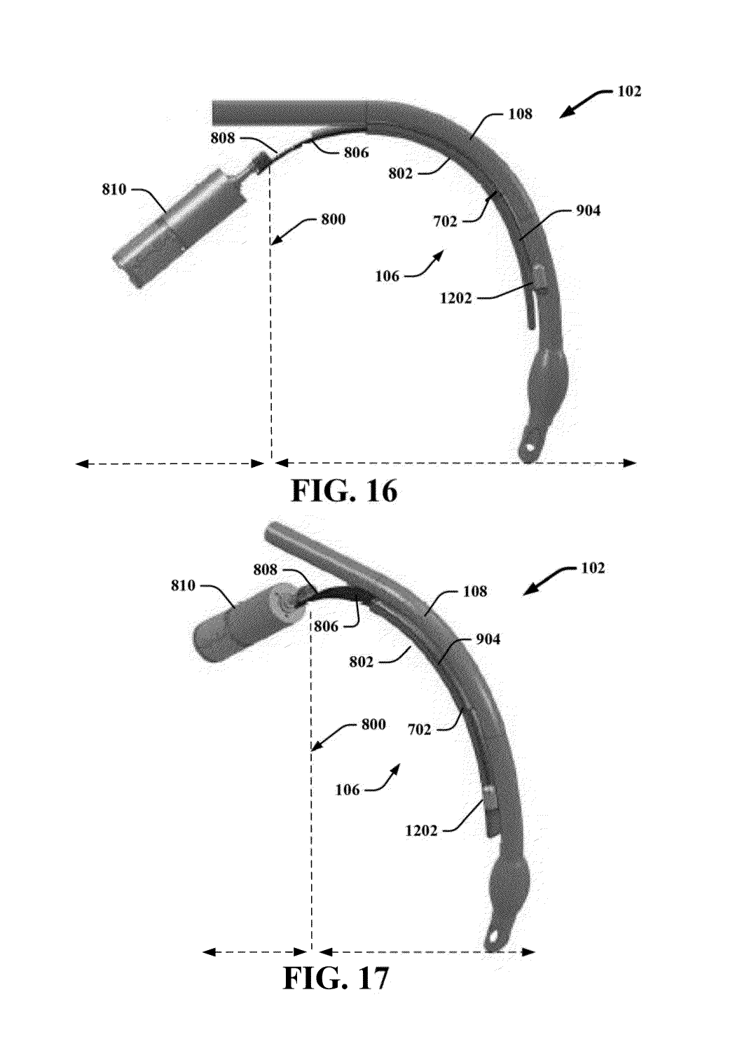

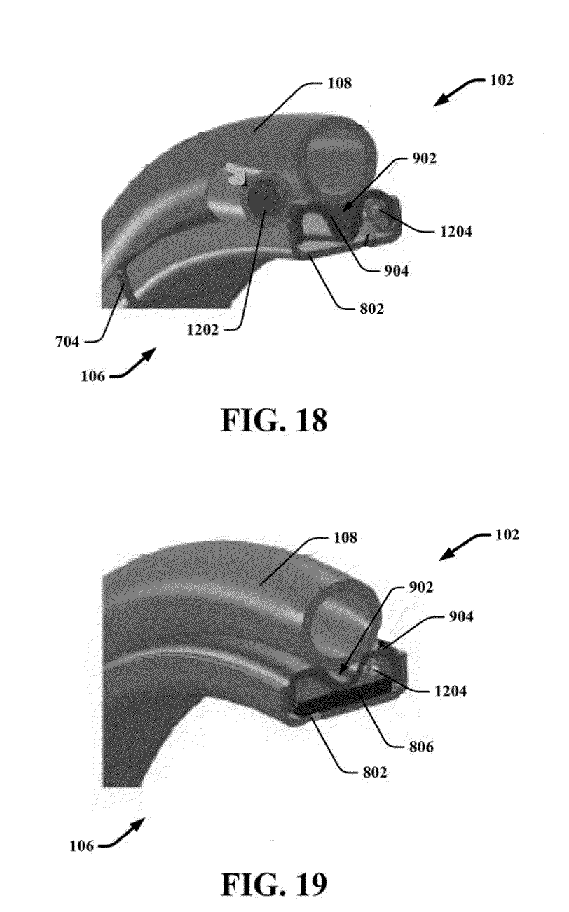

For example, as described in greater detail infra with reference to FIGS. 8-19, the articulating blade 106 can include a channel formed between opposing sidewall railings or retainers. The channel is configured to receive the endotracheal tube 108 (e.g., the endotracheal tube 108 can insert into the channel), and allow for the endotracheal tube to pass there through while holding the endotracheal tube 108 within the channel. In some implementations, movement of the endotracheal tube 108 through the channel is accomplished via a mechanical pushing or advancement mechanism included on the articulating blade 106 and controlled via the handle 104 (e.g., via the control circuit 110). In another implementation, movement of the endotracheal tube 108 through the channel is accomplished via a mechanical mechanism provided on the handle 104 and controlled via the handle 104 (e.g., via the control circuit 110). Still in other implementations, the endotracheal tube 108 can be advanced through the channel manually. The handle 104 can also include a control that releases the endotracheal tube 108 from the articulating blade 106 after proper placement of the endotracheal tube 108 within the patient's trachea. For example, the articulating blade 106 can be configured to grab or otherwise hold the endotracheal tube 108 during the intubation procedure via a latching mechanical mechanism, a pressure mechanism, or another suitable mechanical mechanism. The articulating blade 106 can further be configured to mechanically release hold of the endotracheal tube 108 in response to application of a mechanical and/or electrical signal to the articulating blade 106, via the handle 104, that causes the articulating blade to unlatch the endotracheal tube 108 or otherwise release its grab of the endotracheal tube 108.

The airway management apparatus 102 further includes one or more sensors provided on the articulating blade 106 and/or the endotracheal tube 108. The one or more sensors 112 can be communicatively coupled to the control circuit 110 via a wired or wireless data connection. In some embodiments, the one or more sensors 112 can be communicatively coupled to an external processing device (not shown) via a wired or wireless data connection). The one or more sensors 112 are configured to capture various types of data and provide the captured data to the control circuit 110 for processing, storage, and/or sending to an external device (and/or an external device (not shown)). The one or more sensors can include but are not limited to: image sensors (e.g., a camera configured to capture still images and/or video data), chemical sensors, pressure sensors, temperature sensors, acoustic sensors, motion sensors, and other suitable sensors.

In one or more embodiments, the sensors 112 include one or more cameras that can be maneuvered, via an electrical signal applied by the control circuit 110, in close proximity to the opening of the trachea (e.g., at or near the distal end 118 of the blade and/or at or near the distal end of the endotracheal tube 108). In some implementations, the cameras can move in response to movement of the articulating blade 106 and/or the endotracheal tube 108 via which the cameras are attached. In other embodiments, the cameras can be configured to move independently of the articulating blade 106. For example, a camera located near the distal end 118 of the articulating blade 106 can be configured to change orientation, change field of view, etc., based on electrical signal commands applied thereto via the control circuit 110. The one or more cameras can capture image data (e.g., still images or video image data) in real-time during the intubation procedure.

In some embodiments, the image data captured by the one or more cameras can be displayed to the operator 114 via a display screen (not shown) associated with the airway management apparatus 102 during performance of the intubation procedure, thereby improving the operator's view of the patient's oral cavity and associated anatomy in relation to articulating blade 106 and/or the endotracheal tube 108. For example, image data captured via the one or more cameras can be rendered in real-time via a display provided on the handle 104 (not shown), or a display located at an external device (e.g., a monitor in the operating room, a monitor employed by a remote physician outside the operating room, a heads-up display worn by the operator 114, etc.). When rendered at an external device (not shown), the captured image data can be transmitted to the external device from the airway management apparatus 102 via a wired or wireless data connection. By allowing maneuvering of the one or more cameras, the operator 114 can have an increased chance of appropriately viewing the laryngeal aperture and vocal cords and placing the endotracheal tube 108 into the trachea. Further, the airway management apparatus 102 can provide direct, visual feedback that the endotracheal tube is in a proper place, and thus mitigate adverse events associated with a misplaced endotracheal tube 108.

In an exemplary embodiment, the airway management apparatus includes a plurality of cameras (e.g., two or more than two) that can facilitate generation of a stereoscopic view. Thus, as opposed to conventional techniques where the vocal cords are viewed from outside of the mouth, the cameras can capture a view from the base of the tongue internally. The cameras can be any type of digital cameras including, for instance, charge coupled devices (CCDs) or CMOS sensors that can capture images. The digital camera(s) can be mounted on the articulating blade 106 and moved independently of the articulating blade 106 allowing for improved viewing of the laryngeal opening. These cameras can collect video data and/or still image data. Further, it is contemplated that the cameras can switch between collecting video and still images, simultaneously collect video and still images, or statically collect a particular type of data. Moreover, the cameras can be high definition cameras, for example. Further, the cameras can include a heating element (e.g., coil, light emitting diode, etc.) to mitigate fogging while positioned within the oral cavity.

The one or more sensors 112 can also include a chemical sensor configured to detect a biochemical analyte in the patient's breath, saliva, blood, or other bodily fluid. For example, a chemical sensor can be provided on the articulating blade 106 and/or the endotracheal tube 108 that is configured to detect end-tidal carbon dioxide (EtCO₂) concentration or level. EtCO₂ is the level of carbon dioxide released at the end of an exhaled breath. EtCO₂ reflects cardiac output and pulmonary blood flow as the gas is transported by the venous system to the heart and then pumped to the lungs. A patient's EtCO₂ level increases as the distance between the EtCO₂ sensor and the patient's trachea decreases. Thus correct placement of the articulating blade 106 within the patient's oral cavity and the endotracheal tube 108 within the trachea can be facilitated by monitoring the patient's EtCO₂. For example, the monitored EtCO₂ level can be compared to a threshold or target level associated with proper placement of the articulating blade 106 and/or the endotracheal tube 108. If the monitored EtCO₂ level is at the target or threshold level, the articulating blade 106 and/or the endotracheal tube 108 can be determined to be located in the correct or target position. Further, any other type of property (e.g., pH level, humidity, etc.) can be monitored by one or more sensors 112 to yield similar types of feedback. According to these embodiments, data detected by the chemical sensors (e.g., EtCO₂, PH level, analyte concentration, etc.) can be provided to the control circuit 110 via a wired or wireless connection.

In some embodiments, a chemical sensor can be employed that is configured to change color based on exposure to different levels of a particular analyte. For example, a chemical sensor can be employed that is configured to change color from blue to purple (or other suitable color changes) when it has been exposed to a specific level of EtCO₂ indicative of a target location for placement of the articulating blade 106 and/or the endotracheal tube 108. According to these embodiments, the chemical sensor can be coupled with an image sensor (e.g., a camera) configured to capture image data corresponding to the color of the chemical sensor.

The one or more sensors 112 can also include pressure sensors. For example, a pressure sensor can be provided on or near the pressure cuff (e.g., low pressure cuff 502) to facilitate determining when the pressure cuffhas been properly inflated to establish a seal between the endotracheal tube 108 and the inner wall of the trachea. The pressure sensor can further facilitate monitoring the integrity of the seal between the endotracheal tube 108 and the inner wall of the trachea after the articulating blade 106 has been removed. In another example, a pressure sensor can be provided on the articulating blade 106 to facilitate determining when excessive or insufficient force is encountered in association with contact between the articulating blade 106 and a physical part of the patient's anatomy. For example, the pressure sensor can monitor an amount of pressure applied to depress the patient's tongue or pull back the patient's epiglottis. Similarly, the pressure sensor can monitor when parts of the articulating blade 106 are in contact with a parts of the patient's anatomy when they should or shouldn't be. For example, a pressure sensor provided at the tip of the endotracheal tube 108 can facilitate determining if and when the endotracheal tube 108 is correctly being advanced through the oral cavity and into the trachea based in part on an amount of pressure detected at the tip. According to this example, when pressure is detected at the tip of the endotracheal tube 108, it can be determined that the endotracheal tube 108 is contacting a physical part of the patient's oral anatomy (e.g., the back of the throat, the roof of the mouth, the vocal cords, arytenoids cartilage, etc.) when it shouldn't be.

In various embodiments, data captured by the one or more sensors 112 is processed to facilitate guiding the articulating blade 106 and/or the endotracheal tube 108 to the proper position during intubation. This processing can be performed at the handle 104 via one or more processors associated with the control circuit 110, and/or at an external processing device (not shown) following transmission of the captured sensor data to the external device. For example, image data, analyte sensor data, pressure sensor data, and the like captured via the one or more sensors 112, (collectively referred to as sensor data), can be collected and processed throughout the intubation procedure to identify where and how the articulating blade 106 and/or the endotracheal tube 108 is configured and positioned relative to the various features of the patient's oral anatomy (e.g., the tongue, the epiglottis, the epiglottic vallecula, the laryngeal aperture, the vocal cords, the trachea, etc.). In some embodiments, feedback regarding a current position of the articulating blade 106 and/or the endotracheal tube 108 (and/or a portions thereof) relative to anatomical features of the patient's oral anatomy can be provided to the operator 114 during the intubation procedure via an audible signal (e.g., a beeping sound, an alarm, a speech instruction, etc.) and/or a visual signal (e.g., text on a display, a flashing light, light emission of a particular color, etc.). The operator 114 can interpret the feedback and then adjust the position, orientation, movement, etc., of the articulating blade 106 and/or the endotracheal tube 108 according to the feedback (e.g., via manual movement of the handle 104 and/or the endotracheal tube 108 and/or control features of the handle).

In addition, pressure sensor data can be processed throughout the intubation procedure to determine when excessive, insufficient, or inappropriate force is encountered in association with contact between the articulating blade 106 and/or the endotracheal tube 108 and a physical part of the patient's anatomy. Feedback regarding insufficient, appropriate, inappropriate, and/or excessive pressure can further be provided to the operator 114 of the airway management apparatus 102 via an audible signal (e.g., a beeping sound, an alarm, a speech instruction, etc.) and/or a visual signal (e.g., text on a display, a flashing light, light emission of a particular color, etc.).

In some embodiments, in addition or in the alternative to providing visual and/or audible feedback to the operator 114 regarding positioning and/or maneuvering of the articulating blade 106 and/or endotracheal tube 108 during the intubation procedure, the control circuit 110 can include processing functionality that allows for determining how to correctly position, articulate and maneuver the articulating blade 106 and/or the endotracheal tube 108 during an intubation procedure based on received sensor data and at least one of: known parameters regarding appropriate positioning, maneuvering and pressure application of the articulating blade 106 and/or the endotracheal tube 108 in association with an optimal intubation procedure, known characteristics of a patient's anatomy, a known size, shape and configuration of the articulating blade 106 (at any given point in time), known movement of the articulating blade 106 and/or the endotracheal tube 108 (e.g., rate forward, rate backward, orientation, etc.), and a known distance between the tip of the endotracheal tube 108 and the distal end 118 of the articulating blade 106 (e.g., determined based on a fiducial marker provided on the endotracheal tube 108, a rate of movement of the endotracheal tube through the channel, etc.). Feedback regarding how the operator should move the articulating blade 106 and/or the endotracheal tube can further be provided to the operator. For example, the operator 114 can be provided with feedback regarding how to move or articulate the articulating blade 106 (e.g., articulate X degrees up/down/left/right with fine control, articulate Y degrees up/down/left/right with medium control, extend the blade forward or backwards N millimeters (mm), etc.). The operator can also be provided with feedback regarding how to move the endotracheal tube 108 (e.g., extend forward or backward N mm, move u/down/left/right M mm, etc.).

The control circuit 110 can further automatically articulate and maneuver the articulating blade 106 (e.g., via control of one or more motors included in the handle 104 and physically coupled to the articulating blade 106) to depress the patient's tongue and pull back the patient's epiglottis to reveal the patient's laryngeal aperture and vocal cords. For example, the control circuit 110 can automatically control the mechanical movement (e.g., via one or more motors) of the articulating blade 106 to adjust the curvature of the articulating blade, the length of the articulating blade, the orientation of one or more articulating blade components (e.g., separate blades) at different articulation joints/points, etc. In some implementations, the control circuit 110 can further automatically control advancement of the endotracheal tube 108 through the laryngeal aperture between the vocal cords into a target location within the patient's trachea, in a quick (e.g., less than 3 minutes) and efficient manner with little or no physical irritation or trauma to the patient. In other implementations, the operator 114 can control and manipulate the articulating blade 106 and/or the endotracheal tube 108, and the airway management apparatus 102 can automatically assist and enhance the performance of the operator 114.

The subject airway management apparatus 102 provides a number of advantages as compared to conventional laryngoscopic devices. Every patient has a different anatomically structured airway and securing an airway can be difficult. The airway management apparatus 102 can mitigate such difficulty by producing a reasonable view of the tracheal inlet thereby allowing for placement of an endotracheal tube 108. Additionally, the curvature of the articulating blade 106 can be adapted in real time while in the oral cavity via to accommodate for normal variations in airway anatomy or pathologic airway conditions (e.g., tumors). Such adaptation can be controlled by the operator 114, automatically performed by the airway management apparatus 102 based in part on sensory feedback, and/or controlled via a combined effort of the operator 114 and the airway management apparatus 102. By allowing for variation in the curvature of the articulating blade 106 while within the oral cavity, changing the articulating blade 106 to provide for variations in size and/or shape need not occur (e.g., reducing intubation time, . . . ). Further, trauma to the upper airway can be reduced by employing the airway management apparatus 102 and the physiologic stress on the patient associated with applying force on the tongue and oral cavity tissues can be lessened through a more efficient utilization of force and viewing angles.

Moreover, the ability to visualize the vocal cords is often obstructed by the epiglottis covering the tracheal opening when employing conventional devices. In order to effectively overcome this obstacle, one can place the laryngoscope articulating blade under the epiglottis to bring it out of the way or anteriorly displace the epiglottis by applying anteriorly directed force in the velecula, elevating the epiglottis with the adjoining soft tissue. Traditional laryngoscopes oftentimes fail to do this since to apply anterior force in the velecula requires the operator to “hinge” back on the articulating blade, driving the proximal end of the articulating blade into the patient's incisors. This can result in injury to the teeth, oral mucosa, or cause trauma to the lower part of the airway with adequately improving the view of the tracheal opening. In contrast, the portion of the articulating blade 106 associated with fine control (e.g., tip of the articulating blade 106) can pull the epiglottis out of the way to allow for viewing the vocal cords.

In addition to difficulty associated with visualization of the laryngeal aperture, once the operator obtains a view, it is sometimes difficult to maneuver the endotracheal tube into the trachea to complete the process of securing an airway while employing conventional techniques. The subject airway management apparatus 102 overcomes this drawback by providing an articulating blade 106 that can hold the endotracheal tube 108 and guide the endotracheal tube 108 into the correct position within the trachea.

Furthermore, the subject airway management apparatus 102 can provide real-time feedback regarding positioning and maneuvering of the articulating blade 106 and/or the endotracheal tube 108 throughout the intubation procedure based on data captured via one or more sensors 112. As a result, the operator can more efficiently and effectively perform the intubation procedure. This sensory data can further facilitate automatic robotic maneuvering of the articulating blade 106 and/or the endotracheal tube 108 into the correct positions to mitigate human error associated with performance of the intubation procedure. Moreover, the various features and functionalities of the subject airway management apparatus 102 can be controlled via the handle 104 which can be operated using a single hand.

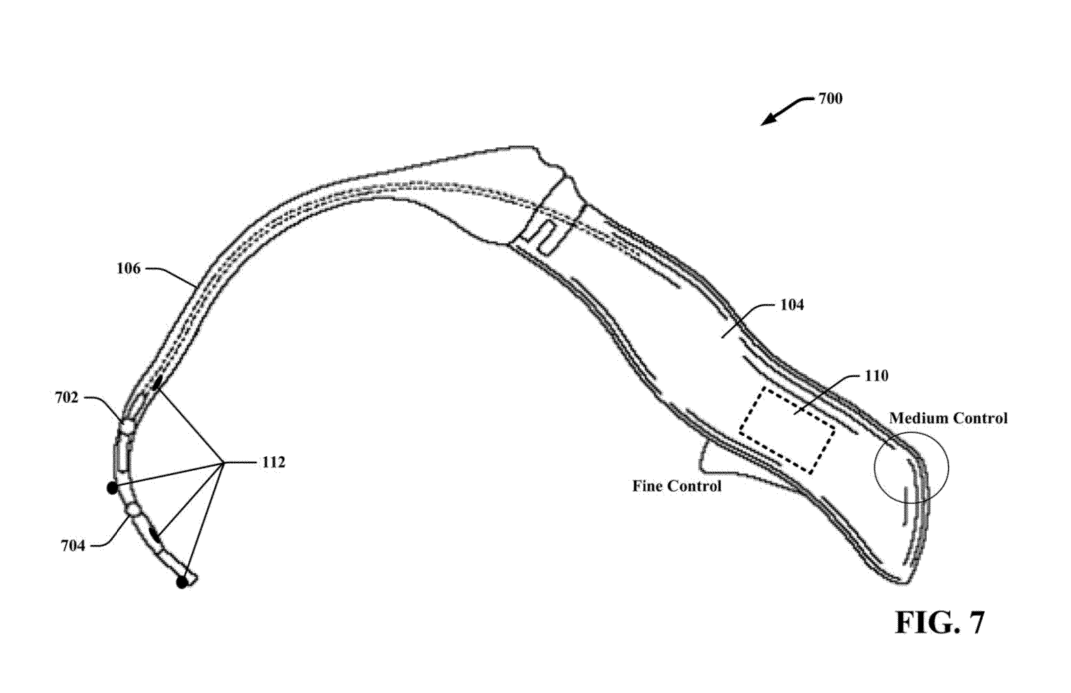

FIG. 7 presents another example airway management apparatus 700 in accordance with one or more embodiments described herein. Airway management apparatus 700 can include same or similar features and functionality as airway management apparatus 102. Repetitive description of like elements employed in respective embodiments described herein is omitted for sake of brevity.

The articulating blade 106 can be a dynamically articulating laryngoscope articulating blade that can be controlled to configure to normal anatomic variants as well as pathologic abnormalities to facilitate placing an endotracheal tube into the trachea. Thus, the articulating blade 106 can accommodate variation in normal and abnormal anatomy of the upper airway resulting in less airway trauma and stimulation stress on a patient undergoing intubation. In contrast to conventional articulating blades that commonly have fixed curvature, the articulating blade 106 can be controlled via the handle 104 to adjust the curvature, manipulate portions or the articulating blade 106 relative to the handle 104, etc. Accordingly, the articulating blade 106 can be slid along the handle 104 to lengthen or shorten the articulating blade. Further, upon obtaining the proper articulating blade length, the articulating blade 106 can be flexed up or down via a medium control articulation point 702 to provide a crude view of the vocal cords (e.g., camera(s) can be positioned nearby the medium control articulation point). Additionally, a tip of the articulating blade 106 can be manipulated via a fine control articulation point 704 to alter the position of a patient's epiglottis to provide a clearer view of the vocal cords. It is contemplated that the articulating blade 106 can be manipulated at any disparate location(s) upon the articulating blade 106 other than or in addition to those depicted in the illustrated schematic.

The articulating blade 106 can be manipulated in any manner. For instance, the size, length, shape, curvature, and the like of the articulating blade 106 or portion(s) thereof can be changed. By way of example, in contrast to some conventional devices with articulating blades that have a fixed curvature, the curvature of the articulating blade 106 can be altered based upon anatomic characteristics of a patient. Further, such adjustments can be effectuated while positioning the airway management apparatus 700 proximate to the trachea within the oral cavity (e.g., as opposed to altering these features while the apparatus is removed from the patient's mouth and thereafter positioning the apparatus). The articulating blade 106 can accommodate variation in normal and abnormal anatomy of the upper airway. Moreover, the articulating blade 106 can reduce airway trauma and stimulation stress on the patient undergoing intubation. Additionally, the articulating blade 106 can be thinner than conventional articulating blades employed in connection with typical laryngoscopic devices.

The articulating blade 106 can have any number of articulation points that can allow for varying degrees of control. For instance, a medium control articulation point 702 can allow for crudely obtaining a view of the vocal cords (e.g., by adjusting an angle of camera(s) to be directed at the vocal cords from the base of the tongue). Further, a fine control articulation point 704 can improve the crude view by manipulating the epiglottis of the patient.

The control circuit 110 can include control components (e.g., blade control component 2002 discussed infra) that enable controlling movement of the articulating blade 106. The control circuit 110 can obtain substantially any type of input to yield a corresponding alteration of the articulating blade 106. For example, the control circuit 110 can receive an input from a user of the airway management apparatus 102 (e.g., via a button, joystick, switch, lever, touch screen, voice command, sensor, mouse, trigger, etc). According to another illustration, an input can be provided from a remotely located user via a signal; thus, telemedicine can be performed such that a user other than a user physically touching the airway management apparatus 102 can provide input utilized to manipulate the articulating blade 106. Moreover, the control circuit 110 can adjust the articulating blade 106 mechanically, via an electrical signal, and so forth.

By way of illustration, the input can be utilized to control one or more motors to manipulate the articulating blade 106. For instance, servo motor(s) can leverage the input to smoothly control movement of the articulating blade 106 in substantially any number of planes. Additionally or alternatively, linear motor(s) can employ the input to manipulate the articulating blade 106. Thus, according to an example, the control circuit 110 can receive a user input, which can control servo motor(s) and/or linear motor(s) that can elongate, shorten, alter elevation, etc. associated with the articulating blade 106 or a portion thereof.

The articulating blade 106 can also have one or more digital cameras (e.g., stereoscopic cameras) mounted thereupon (e.g., one or more of sensors 112). The digital camera(s) can be moved independently of the articulating blade 106, for instance, to allow for optimal viewing of the laryngeal opening. Further, articulation of the articulating blade 106 can enable positioning the camera(s) such that an unobstructed view of the vocal cords can be obtained. It is to be appreciated that the camera(s) can be integrated into the articulating blade 106, attached to the articulating blade 106 (e.g., permanently or temporarily), and so forth. According to an example, the camera(s) can be removeably attached to the articulating blade 106 thereby allowing for replacement.

According to another example, a sleeve-type cover (not shown) can be placed over the articulating blade 106 and/or the handle 104 to enable reuse of the device without cleaning. According to an illustration, the sleeve-type cover can be disposable; however, it is to be appreciated that the cover can be sterilized to allow for reuse of the cover. Moreover, the cover can allow for the articulating blade 106 to be articulated as well as data to be collected (e.g., via the cameras attached to the articulating blade 106) while mitigating obstruction thereof.

The articulating blade 106 can also include a light transmission component (not shown) that can illuminate a patient's airway. For instance, controls (e.g., that alter on/off state, intensity, direction, wavelength, etc.) for the light transmission component can be included in the handle 104 of the airway management apparatus 700. Moreover, the light transmission component can be permanently affixed to, incorporated into, temporarily attached to (e.g., removable, replaceable, etc.) the articulating blade 106.

Further, the articulating blade 106 can include an airway atomizing device, which can be used to deliver topical anesthesia during placement of an endotracheal tube. For example, in certain situations, patients may present with a physical exam that deems them as very challenging airways because of anatomic changes or pathologic tumors. In these situations, patients may need to have their airways secured without the addition of any anesthetic medications that may lead to sedation and a cessation of breathing or an obstruction of the patent airway that they initially presented with making things more urgent and often more difficult and stressful on the patient. Applying local anesthetics to these specific airways allows for the anesthesiologist to place a fiberoptic camera or gently place a laryngoscope to determine if it is safe to place the patient asleep prior to placing a breathing endotracheal tube. The airway management apparatus 700 can have a channel that operates using Bernoulli principles to atomize liquid local anesthetic medications. This coupled with the camera system can allow one to completely topicallize the airway while the device is being placed resulting in a much more comfortable state of the patient as well as maintaining a spontaneously breathing state.

FIGS. 8-19 provide various enlarged perspectives of example features of airway management apparatus 102 in accordance with one or more embodiments. Airway management apparatus 102 can include same or similar features as airway management apparatus 700. Repetitive description of like elements employed in respective embodiments herein is omitted for sake of brevity.

As shown in FIGS. 8 and 9, airway management apparatus 102 includes an articulating blade 106 (indicated by the portion of airway management apparatus 102 to the right of dashed line 800) and a handle portion (indicated by the portion of the airway management apparatus to the left of dashed line 800). The articulating blade 106 includes a base blade 802 having sidewall railing or retainer segments 804 formed thereon. A channel 902 (as shown in FIG. 9) is formed between opposing sidewall railing or retainer segments 804. The retainer segments 804 are formed on different segments of the blade defined or separated by articulation points 702 and 704. Portions of the articulating blade 106 where the articulation points 702 and 704 are located do not include retainer segments 804 formed thereon. The channel 902 is configured to receive an endotracheal tube (e.g., the endotracheal tube 108 can insert into the channel 902), and allow for the endotracheal tube to pass there through while holding the endotracheal tube within the channel. In an aspect, the channel 902 can accommodate endotreacheal tubes of different sizes. For example, the retainer segments 804 can be flexible. The channel 902 can allow for secure and directional placement of variously sized endotracheal tubes, intubating stylets, jet ventilation equipment, and the like. The channel 902 can be employed to facilitate passing an endotracheal tube into the trachea under direct vision, for example.

The articulating blade 106 further includes an upper blade 904 formed on the base blade 802. The upper blade 904 is affixed to the base blade 802. Accordingly the base blade 802 and the upper blade 904 are configured to move together as a single unit. For example, the base blade 802 and the upper blade 904 are configured to bend or articulate together at the articulation joins 702 and 704. The base blade 802 and the upper blade 904 are also configured to move forward and/or backward as a single unit. The articulating blade 106 also includes a pusher piece 806 that is provided between the base blade 802 and the

[symbol]

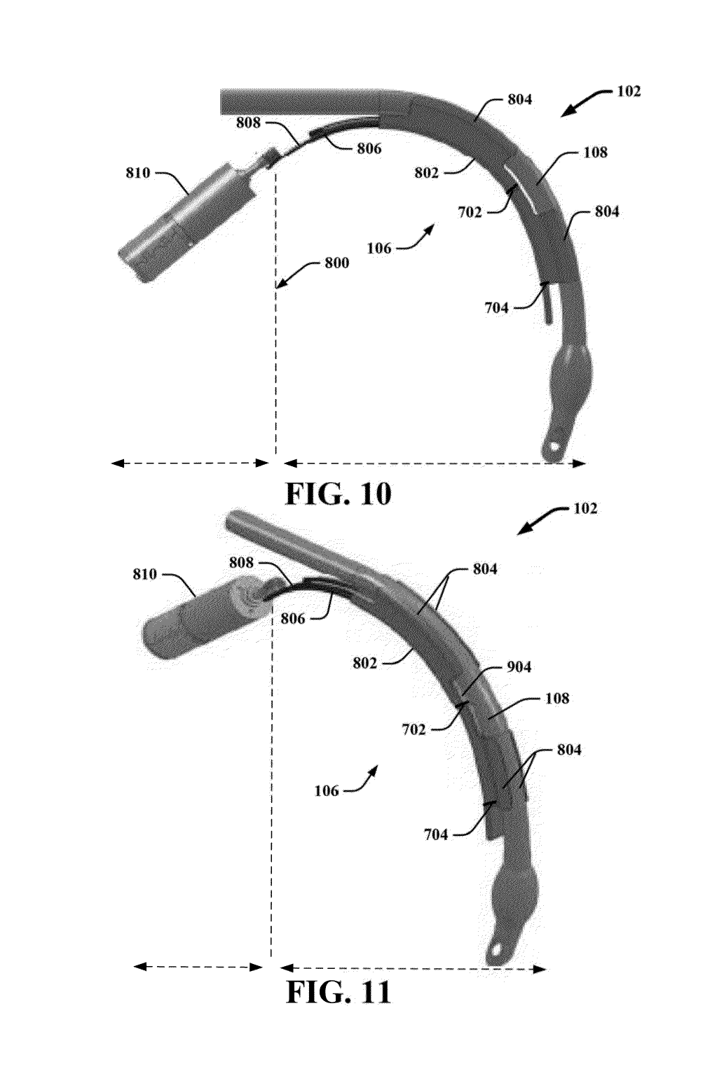

FIGS. 10 and 11 present the airway management apparatus 102 with the endotracheal tube 108 provided in the channel 902. When provided in the channel 902, the endotracheal tube 108 lies adjacent to the upper blade 904. In some implementations, the endotracheal tube 108 is pushed through the channel and advanced into a patient's trachea manually. In other implementations, movement of the endotracheal tube 108 through the channel is accomplished via a mechanical pushing or advancement mechanism included on the articulating blade 106 and controlled via the handle (not shown). In another implementation, movement of the endotracheal tube 108 through the channel is accomplished via a mechanical pushing mechanism provided on the handle and controlled via the handle (not shown). According to this implementation, the endotracheal tube 108 can integrate with the mechanical pushing mechanism provided on the handle. The handle 104 can also include a control that releases the endotracheal tube 108 from the articulating blade 106 after proper placement of the endotracheal tube 108 within the patient's trachea. For example, the retainer segments 804 can be configured to expand in response to application of a mechanical and/or electrical signal thereto, and release the endotracheal tube 108.

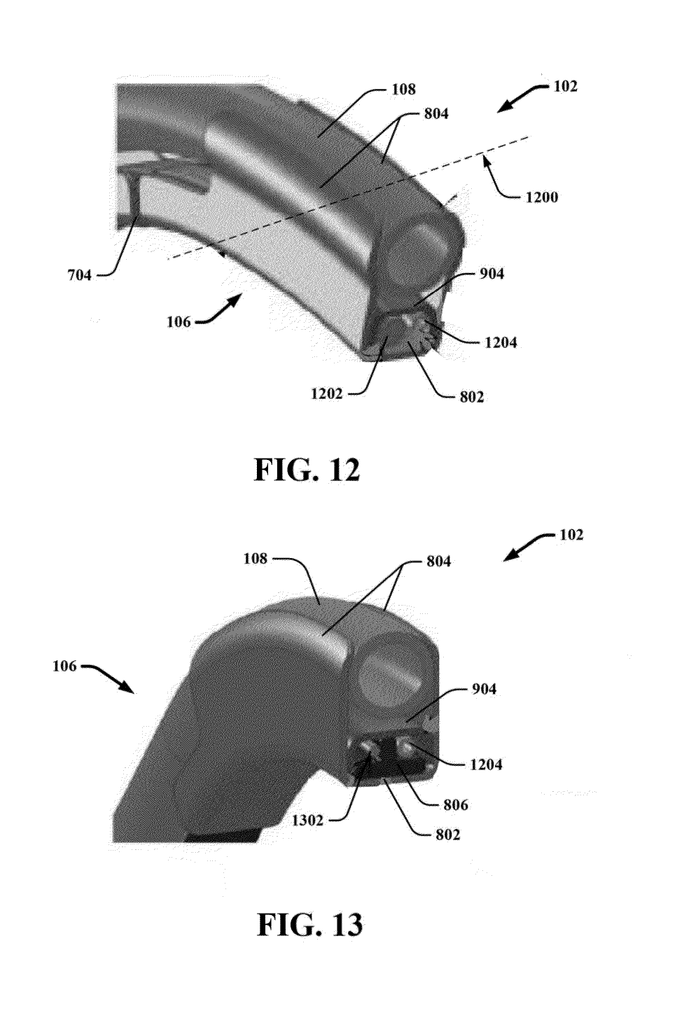

FIGS. 12 and 13 present an enlarged perspective of the distal end (e.g., distal end 118) of the airway management apparatus 102. FIG. 13 depict a cross-sectional view of airway management apparatus when sliced vertically along dashed line 1200 in FIG. 12. As shown in FIG. 12, the distal tip of the articulating blade includes a camera 1202 and a distal blade control cable 1204. The camera 1202 is operated via a camera control cable 1302 connected thereto.

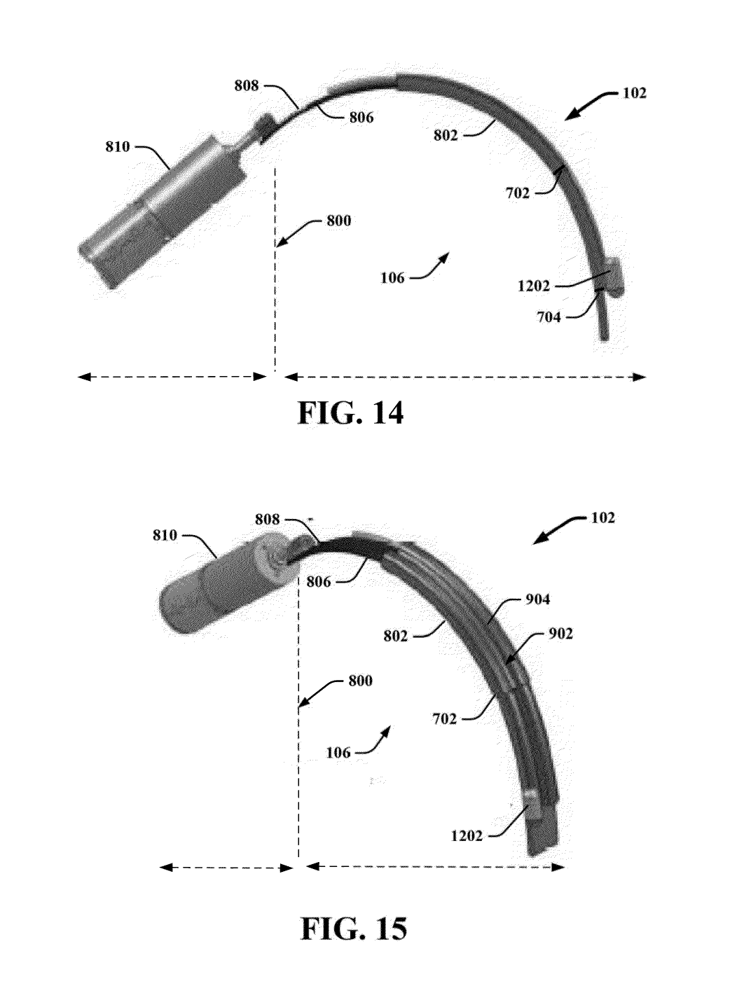

FIGS. 14-19 present various perspectives of airway management apparatus 102 with the retainer segments 804 removed in accordance with one or more embodiments described herein. In some implementations, the upper blade 904 has a non-planar surface topology. For example, as shown in FIGS. 14 and 17-19, the middle or center portion of the upper blade can have a dip or valley shape that contours to the rounded surface of the endotracheal tube 108 provided thereon. In some implementations, the upper blade 904 can grip or hold the endotracheal tube in place without the need of retainer segments 804.

In the various embodiments of airway management apparatus 102 depicted in FIGS. 8-19, the endotracheal tube 108 is configured to lie on and adjacent to the upper blade 904 which is formed on and adjacent to an upper surface of the base blade 802. With this configuration, when the airway management apparatus 102 is employed to facilitate intubation, the bottom surface of the base blade 802 is configured to touch the patient's tongue and epiglottis and the endotracheal tube is configured to move through the patient's oral cavity directly over the base blade 802.

However, in one or more additional embodiments, the articulating blade 106 can be configured with a channel (e.g., channel 902) formed on a side surface of the articulating blade 106. For example, the articulating blade 106 depicted in FIGS. 8-19 can be rotated 180° clockwise or counter clockwise such that the side surfaces of the retainer segments 804 on the right or left side of the articulating blade 106 are configured to touch the patient's tongue during intubation. With this configuration, the side surfaces of the retainer segments 804 on the right or left side of the base blade 802 can be formed as a continuous or substantially continuous piece of material to function as a depressor. Alternatively, an additional base blade (not shown) can be included on the articulating blade 106 that is coplanar or substantially coplanar with the side surfaces of the retainer segments and perpendicular to the base blade 802. For example, the articulating blade 106 can have the shape of an upside letter T, wherein the base blade 802 is configured to be perpendicular or substantially perpendicular to the tongue during intubation. This additional base blade is configured to touch and depress the patient's tongue during intubation. According to this embodiment, the endotracheal tube 108 is advanced through the patient's oral cavity on a left or right side of the base blade 802 and the additional base blade.

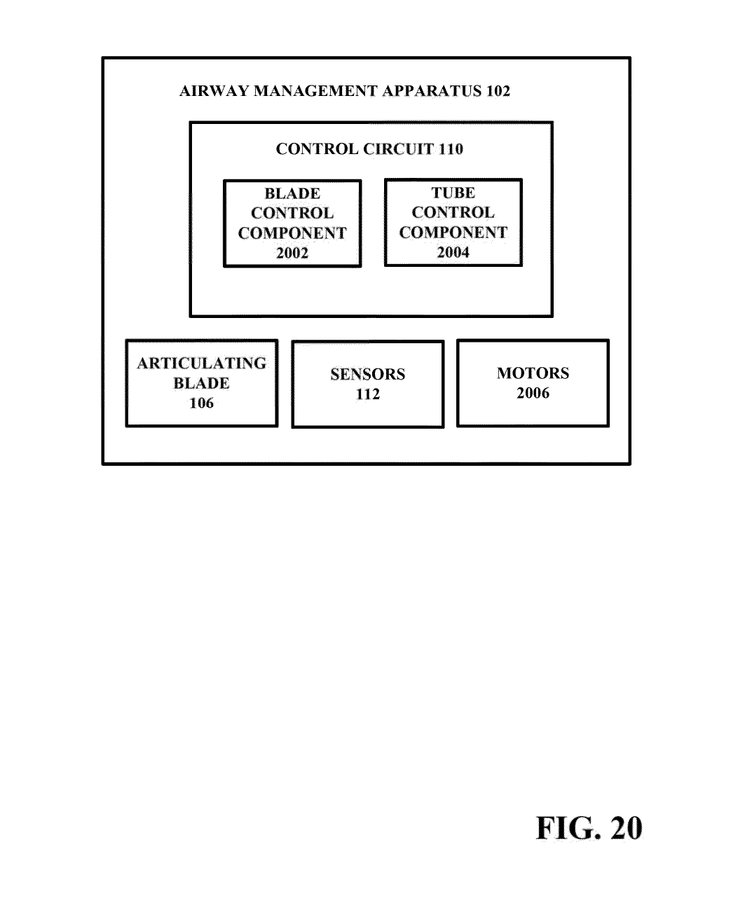

With reference now to FIG. 20, presented is a high-level block diagram of example airway management apparatus 102 in accordance with one or more embodiments described herein. Airway management apparatus 102 can include same or similar features and functionality as airway management apparatus 700 and the like. Repetitive description of like elements employed in respective embodiments described herein is omitted for sake of brevity.

Embodiments of devices, apparatus and systems herein can include one or more machine-executable components embodied within one or more machines (e.g., embodied in one or more computer-readable storage media associated with one or more machines). Such components, when executed by the one or more machines (e.g., processors, computers, computing devices, virtual machines, etc.) can cause the one or more machines to perform the operations described.

With reference to FIGS. 1 and 20, airway management apparatus 102 includes control circuit 110, articulating blade 106, one or more sensors 112 and one or more motors 2006. One or more components of airway management apparatus 102 can be provided at various locations of the on or within the apparatus. For example, in one embodiment, the control circuit 110 is provided within a housing of a handle (e.g., handle 104) of the apparatus to which the articulating blade 106 is attached. The one or more sensors 112 can be provided at various locations on the articulating blade 106. In an aspect, one or more sensors 112 can be provided on the endotracheal tube 108 that is guided into place within a patient's trachea via the airway management apparatus 102 and/or manually. The control circuit 110 is communicatively coupled (e.g., via a wired connection or a wireless connection) to the articulating blade 106 and/or various mechanical and electrical components of the articulating blade 106 (e.g., parts of the one or more motors 2006, the one or more sensors 112, etc.).

The one or more motors 2006 can include for example, a servo-motor, a linear motor, or another suitable motor. A servomotor is a packaged combination of several components, including but not limited to: a motor (e.g., an electric motor as applied to airway management apparatus 102), a gear train to reduce the many rotations of the motor to a higher torque rotation, a position encoder that identifies the position of the output shaft and an inbuilt control system. The input control signal to the servo indicates the desired output position. A linear motor is an electric motor an electric motor that produces straight-line motion (as opposed to rotary motion) by means of a linear stator and rotor placed in parallel.

In various embodiments, at least one of the motors 2006 is physically and/or electrically coupled (e.g., via one or more wires) to the control circuit 110 and one or more parts of the articulating blade 106. In some embodiments, the motor is provided within or substantially within the housing of the handle 104. However, depending on type of motor and employed and the type of movement of the articulating blade the motor is configured to control, one or more components of the motor can be located on or within the articulating blade 106. For example, a motor physically and/or electrically coupled to one or more parts of the articulating blade 106 and the control circuit can be configured to control movement of the articulating blade forward, backward, left, and right relative to the handle 104. The motor or anther motor can also control articulation of the articulating blade 106 (in substantially any direction) at one or more articulation points of the articulating blade 106 (e.g., medium control articulation point 702, and fine control articulation point 704). Articulation at the different articulation points can further be controlled independently.

In some embodiments, at least one of the one or more motors is physically and/or electrically coupled to the endotracheal tube 108 and the control circuit 110. For example, the output shaft of the motor can be physically coupled to the endotracheal tube 108 and configured to drive the endotracheal tube 108 forwards and backwards within a channel of the articulating blade (e.g., channel 902). In some aspects, the motor configured to control movement of the endotracheal tube is provided entirely on or within the handle 104. However, in other aspects one or more portions of the motor can be located on or within the articulating blade 106.

In addition, a motor of the one or more motors 2006 can be physically and/or electrically coupled to a mechanical component on or within the handle and/or the articulating blade that is responsible for holding the endotracheal tube 108 within the channel and releasing the endotracheal tube 108 from the channel (e.g., the retainer segments 804, the upper blade 904, or another suitable mechanical component provided on or within the handle 104 and/or the articulating blade 106). The motor can be configured to control the mechanical component to facilitate holding the endotracheal tube 108 within the channel during intubation and releasing the endotracheal tube from the channel and the articulating blade 106 after successful placement of the endotracheal tube 108 within the trachea.

The control circuit 110 includes blade control component 2002 to facilitate controlling mechanical and/or electrical functions of the articulating blade 106 (e.g., movement of the articulating blade 106, data capture via sensors 112, etc.). The blade control component 2002 can include hardware, software, or a combination of hardware and software. The blade control component 2002 is configured to receive input corresponding to a movement or motion of the articulating blade 106 or a part of the articulating blade (e.g., an articulation joint, an articulating blade segment, etc.). The input can define various types of movement and/or parameters associated with the movement (e.g., speed, distance, rotational degree, etc.). The blade control command is configured to interpret the input, and cause, via an electrical signal, a motor physically coupled to the articulating blade 106 and/or the part of the articulating blade to perform the movement. For instance, the movement can include articulation of one or more segments of the blade via the one or more articulation points in substantially any direction, elongation of the articulating blade, shortening of the articulating blade, elevation of the articulating blade, etc., or a portion thereof.

The input received by the blade control component 2002 can be provided in various manners. In some embodiments, the handle 104 can include input devices or components that provide the input based on user interaction therewith. For example, the input devices or components can include physical buttons, joysticks, roller balls, etc., provided on the handle 104 that can be pressed or moved by the user in a particular manner that corresponds to a defined movement. In another example, the input devices or components can include a touchscreen, a mouse, a keyboard, a motion controlled input device or a voice controlled input device. Interaction of the user with the input devices can provide varying degrees of control precision (e.g., medium control, fine control, etc.) of the articulating blade 106. In one or more embodiments, these input devices can be located at a remote device (e.g., remote airway management device 2602 discussed infra). According to these embodiments, the input received via the input devices is converted to a control signal that can be transmitted or sent the airway management apparatus 102 and interpreted and applied by the blade control component 2002.

In some embodiments, the control circuit 110 can also include a tube control component 2004 to control mechanical and/or electrical functions of an endotracheal tube (e.g., endotracheal tube 108) that is partially and removably attached to the airway management apparatus 102. For example, the tube control component 2004 can facilitate guiding or moving the intubation endotracheal tube into a patient's trachea via the airway management apparatus 102 based on received input. The input can be provided and/or received in the various manners discussed with respect to blade control component 2002. In other embodiments, the endotracheal tube can be manually moved into place within the patient's trachea. The tube control component 2004 and/or the blade control component 2002 can also control retaining and releasing the endotracheal tube from the channel based in received input. When one or more sensors 112 are provided on the endotracheal tube 108, the tube control component 2004 can also control data capture via the one or more sensors.

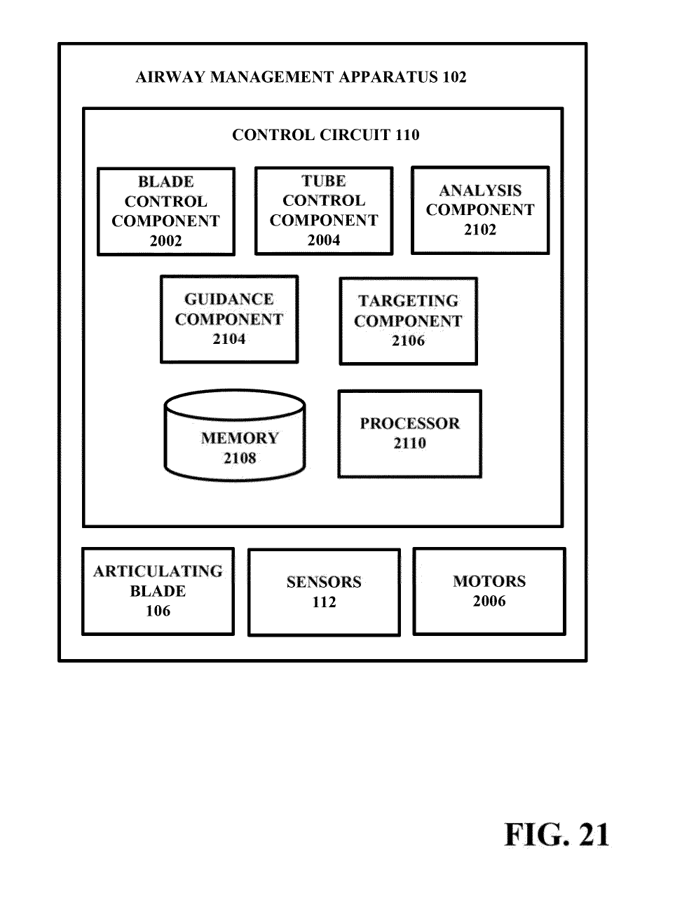

FIG. 21 presents is a high-level block diagram of example airway management apparatus 102 in accordance with one or more additional embodiments described herein. Repetitive description of like elements employed in respective embodiments described herein is omitted for sake of brevity.

As shown in FIG. 21, the control circuit 110 of airway management apparatus 102 can also include analysis component 2102, guidance component 2104, targeting component 2106, memory 2108, and processor 2110. One or more of the components of airway management apparatus 102 constitute machine-executable component(s) embodied

[symbol]

With reference to FIGS. 1 and 21, analysis component 2102 is configured to aggregate and evaluate the sensor data obtained by the one or more sensors 112. For example, one or more sensors 112 can provide input sensor data to the analysis component 2102 which can thereafter aggregate such input data to yield a unified output. In various embodiments, analysis component 2102 is configured to analyze data captured by the one or more sensors 112 to facilitate guiding the articulating blade 106 and/or the endotracheal tube 108 to the proper position during intubation. For example, an ideal intubation procedure involves depressing the patient's tongue, pulling back the patient's epiglottis with the articulating blade 106 to reveal the patient's laryngeal aperture and vocal cords, and inserting the endotracheal tube 108 through the laryngeal aperture between the vocal cords into the patient's trachea, in a quick (e.g., less than 3 minutes) and efficient manner with little or no physical irritation or trauma to the patient. As previously discussed, this task is difficult to achieve do to variants in patient anatomy, complications (e.g., inability to visualize the laryngeal aperture, malposition, aspiration, hypoxia, larngospas, oropharnygeal trauma, vagal stimulation, etc.), and the configuration and capabilities of the conventional laryngoscope articulating blade.

With the subject airway management apparatus 102, image data, analyte sensor data, pressure sensor data, and the like captured via the one or more sensors 112, (collectively referred to as sensor data), can be received and processed by the analysis component 2102 throughout the intubation procedure to identify where and how the articulating blade 106 and/or the endotracheal tube 108 is configured and positioned relative to the various features of the patient's oral anatomy (e.g., the tongue, the epiglottis, the epiglottic vallecula, the laryngeal aperture, the vocal cords, the trachea, etc.). For example, the analysis component 2102 can employ pattern recognition to correctly identify and characterize different anatomical features represented by image data received from one or more imaging sensors (e.g., cameras). For example, using information stored in memory 2108 (or otherwise accessible to analysis component 2102) that correlates specific patterns in image data with known anatomical features and/or points of the anatomical features, analysis component 2102 can identify image data corresponding to a location on the upper surface of the tongue, a location on the lower surface of the tongue, an inner lining of the cheek, an upper surface or the epiglottis, the epiglottic vallecula, a vocal cord or the vocal cords, the laryngeal aperture, etc.

The analysis component 2102 can further determine the three-dimensional position of a camera provided on the articulating blade 106 or the endotracheal tube 108 from which the image data was received, (wherein the camera position corresponds to a known location of a part of the articulating blade 106 or the endotracheal tube 108), relative to various anatomical features of the patient's oral using depth information captured via the camera in association with two-dimensional with image data captured via the camera. In another example, the analysis component 2102 can determine the three-dimensional position of the camera relative to various anatomical features within the patient's oral cavity based on a know relationship between the camera position and a fiducial marker represented in the image data located on the airway management apparatus 102 and/or the patient. In another example, the analysis component 2102 can determine the three-dimensional position of a camera relative to various anatomical features within the patient's oral cavity can be determined based comparison of image data captured from two or more cameras having known positions and orientations on the articulating blade 106 or the endotracheal tube 108 at the time of data capture.

In addition, the analysis component 2102 can determine the position of the articulating blade 106 or the endotracheal tube 108 relative to various anatomical features of the patient based on captured EtCO₂ levels throughout the intubation process, wherein a particular level is associated with a distance between the articulating blade 106 and/or the endotracheal tube 108 and a target position within the trachea. Further, the analysis component 2102 can determine the position and orientation of the articulating blade 106 and/or the endotracheal tube 108 relative to various anatomical features of the patient based on pressure sensor data identifying amounts of pressure applied to various points of the articulating blade 106 and/or the endotracheal tube 108 due to physical contact of the articulating blade 106 and/or the endotracheal tube 108 with internal parts of the patient. For example, pressure sensor data indicating X amount of pressure is being received at a sensor located on a right sidewall of a particular retainer segment (e.g., of retainer segments 804) can indicate that segment is touching the inner wall of the patient's right cheek, a left side of the patient's tongue, etc.).

Accordingly, the analysis component 2102 can accurately determine a current position and orientation of the articulating blade 106 and/or the endotracheal tube 108 (and/or a portions thereof) relative to anatomical features of the patient's oral anatomy throughout the intubation procedure based on combined information including but not limited to: the sensor data described above, known characteristics of a patient's anatomy (e.g., where features are located relative to one another, data correlating image patterns with features and/or points of the features, etc.), a known size, shape and configuration of the articulating blade 106 (at any given point in time), and known movement of the articulating blade 106 and/or the endotracheal tube 108 (e.g., rate forward, rate backward, orientation, etc.). For example, a three-dimensional position of the articulating blade 106 and/or the endotracheal tube 108 can be confirmed or calibrated based on captured image data combined with chemical sensor data and/or pressure sensor data and a known configuration or shape of the airway management apparatus 102. The analysis component 2102 can also determine a current position of the endotracheal tube 108 relative to anatomical features of the patient's oral anatomy throughout the intubation procedure based on and a known distance between the tip of the endotracheal tube 108 and the distal end 118 of the articulating blade 106 (e.g., determined based on a fiducial marker provided on the endotracheal tube 108 included in captured image data, determined based on a rate of movement of the endotracheal tube through the channel, etc.).

In some embodiments, feedback regarding a current position of the articulating blade 106 and/or the endotracheal tube 108 (and/or a portions thereof) relative to anatomical features of the patient's oral anatomy can be provided to the operator 114 during the intubation procedure. For example, as noted above, image data captured via one or more cameras can be presented to the operator via a display (e.g., located on the handle 104 and/or at an external device). In addition, image data can be generated and presented to the operator that displays the articulating blade 106 and/or the endotracheal tube 108 in its current position, configuration, and orientation relative to the various internal physical features of the patient. According to these embodiments, guidance component 2104 can facilitate generating and/or rendering such image data.

Guidance component 2104 can also generate (or facilitate generating at an external device) text or audible speech feedback identifying a current position of the articulating blade 106 and/or the endotracheal tube 108 relative to one or more anatomical features of the patient. The text feedback can be presented via a display (e.g., located at the airway management apparatus or an external device) and the audible feedback can be rendered via a speaker located at the airway management apparatus (e.g., in the handle 104) or an external device. For example, text can be provided to the operator 114 on a display or a spoken instruction can be generated via a speaker that indicates a current distance between the articulating blade 106 and the epiglottis and/or a current distance between the distal end of the endotracheal tube 108 and a target position within the patient's trachea. The operator 114 can then adjust the position, orientation, movement, etc., of the articulating blade 106 and/or the endotracheal tube 108 according to the feedback.

In addition, analysis component 2102 can analyze pressure sensor data throughout the intubation procedure to determine when excessive, insufficient, or inappropriate force is encountered in association with contact between the articulating blade 106 and/or the endotracheal tube 108 and a physical part of the patient's anatomy. For example, the one or more sensors 112 can include a pressure sensor configured to monitor an amount of pressure applied to depress the patient's tongue, pull back the patient's epiglottis, or otherwise encountered in association with contact between the articulating blade 106 and/or the endotracheal tube 108 and the patient. The monitored pressure sensor data can be compared with known benchmarks or thresholds (e.g., stored in memory 2108 or otherwise accessible to analysis component 2102) to determine when an appropriate or inappropriate amount of pressure is applied.

Similarly, the pressure sensor data can be processed to determine when parts of the articulating blade 106 and/or the endotracheal tube 108 are in contact with parts of the patient's anatomy when they should or shouldn't be, thereby causing unnecessary irritation or trauma. For example, the analysis component 2102 can employ information stored in memory 2108, or otherwise accessible to the analysis component 2102, that correlates pressure sensor readings from pressure sensors located at different positions on or within the articulating blade 106 with appropriate or inappropriate contact of certain anatomical features or points of the anatomical features throughout the intubation procedure. The analysis component 2102 can also determine a particular anatomical feature or point on an anatomical feature that is in contact with a particular point or part of the articulating blade 106 (wherein the pressure sensor is located at the particular point of the articulating blade) based on a determination regarding a current position and orientation of the articulating blade relative to the anatomical feature. Guidance component 2104 can also generate and provide feedback to the operator 114 of the airway management apparatus 102 regarding insufficient, appropriate, inappropriate, and/or excessive pressure via an audible signal (e.g., a beeping sound, an alarm, a speech instruction, etc.) and/or a visual signal (e.g., text on a display, a flashing light, light emission of a particular color, etc.).

In some embodiments, in addition or in the alternative to providing visual and/or audible feedback to the operator 114 regarding positioning and/or maneuvering of the articulating blade 106 and/or endotracheal tube 108 during the intubation procedure, the control circuit 110 can include processing functionality that allows for determining how to correctly position, articulate and maneuver the articulating blade 106 and/or the endotracheal tube 108 during an intubation procedure based on received sensor data and at least one of: know parameters regarding appropriate positioning, maneuvering and pressure application of the articulating blade 106 and/or the endotracheal tube 108 in association with an optimal intubation procedure, known characteristics of a patient's anatomy, a known size, shape and configuration of the articulating blade 106 (at any given point in time), known movement of the articulating blade 106 and/or the endotracheal tube 108 (e.g., rate forward, rate backward, orientation, etc.), and a known distance between the tip of the endotracheal tube 108 and the distal end 118 of the articulating blade 106 (e.g., determined based on a fiducial marker provided on the endotracheal tube 108, a rate of movement of the endotracheal tube through the channel, etc.).

For example, control circuit 110 can include targeting component 2106 to facilitate guiding or bringing the articulating blade 106 and/or the endotracheal tube to a target location within the patient's oral cavity and/or trachea, respectively. For example, as described above, analysis component 2102 can determine a current location and orientation of the articulating blade 106 and/or the endotracheal tube relative to a particular anatomical feature or point of the anatomical feature. In one or more embodiments, target locations for placement of the articulating blade 106 and/or the endotracheal tube 108 within the oral cavity and/or the trachea, respectively, are predetermined. For example, a first target location for the distal end (e.g., distal end 118) of the articulating blade 106 can be the epiglottic vallecula and second target location for the distal end of the endotracheal tube 108 can be N centimeters past the vocal cords.

Based on a current position and orientation of the articulating blade 106 and the location of the first target location, the targeting component 2106 can determine a first trajectory path for safely and correctly moving the articulating blade 106 to the first target location. Further, based on a known size, shape, and configuration of the articulating blade 106 and the determined first trajectory path, the targeting component 2106 can determine one or more movements for the articulating blade 106 that effectuate bringing the articulating blade to the first articulation point along the first trajectory path. For example, the targeting component 2106 can determine when and how to orient the articulating blade, and how to articulate the articulating blade via one or more articulation points in association with moving the articulating blade a particular distance forward, backward, upward or downward. For instance, the targeting component 2106 may determine the articulating blade should be articulated X degrees up/down/left/right with fine control, articulated Y degrees up/down/left/right with medium control, extended forward or backwards N millimeters (mm), etc.

Similarly, based on a current position and orientation of the endotracheal tube 108 and the location of the second target location, the targeting component 2106 can determine a second trajectory path for safely and correctly moving the endotracheal tube 108 to the second target location. Further, based on a known size (e.g., length and diameter) of the endotracheal tube 108 and the determined second trajectory path, the targeting component 2106 can determine one or more movements for the endotracheal tube 108 that effectuate bringing the endotracheal tube 108 to the second target location along the second trajectory path. For example, the targeting component 2106 can determine when and how far to extend the endotracheal tube 108 forward or backward, and up/down/left/right.

In various embodiments, the guidance component 2104 is further generate and provide feedback regarding how to move, orientate, and articulate the articulating blade 106 and/or the endotracheal tube 108 as determined via the targeting component 2106. For example, the guidance component 2104 can generate an image data output, a textual output, an audible output, etc., that provides specific instruction regarding the determined movements for the articulating blade 106 and/or the endotracheal tube 108. For example, the operator 114 can be provided with feedback regarding how to move or articulate the articulating blade 106 (e.g., articulate X degrees up/down/left/right with fine control, articulate Y degrees up/down/left/right with medium control, extend the blade forward or backwards N millimeters (mm), etc.). The operator can also be provided with feedback regarding how to move the endotracheal tube 108 (e.g., extend forward or backward N mm, move up/down/left/right M mm, etc.).

In some implementations, the guidance component 2104 can also generate (e.g., via a speaker at located in the handle 104), or initiate generation (e.g., at an external device), audible signals that indicate when the articulating blade 106 and/or the endotracheal tube 108 is in the correct or incorrect position and/or configuration and/or when the articulating blade 106 and/or the endotracheal tube 108 are being advanced/moved in a correct or incorrect trajectory. For example, a high pitched beeping signal can be used to indicate the articulating blade 106 and/or the endotracheal tube 108 is being manipulated in a correct trajectory while a low pitched beeping signal can be used to indicate the articulating blade 106 and/or the endotracheal tube 108 is being manipulated in an incorrect trajectory. The rate of the beeping signal can also vary to indicate a distance between the distal end 118 of the articulating blade 106, and/or a distal end of the endotracheal tube 108, to a target location. For example, a target location for the distal end 118 of the articulating blade 106 is generally the epiglottic vallecula and a target location for the distal end of the endotracheal tube 108 is generally a few centimeters past the vocal cords. The rate of the beeping signal can increase as the distal end 118 of the articulating blade 106 approaches the target position and/or as the distal end of the endotracheal tube 108 approaches the target position.

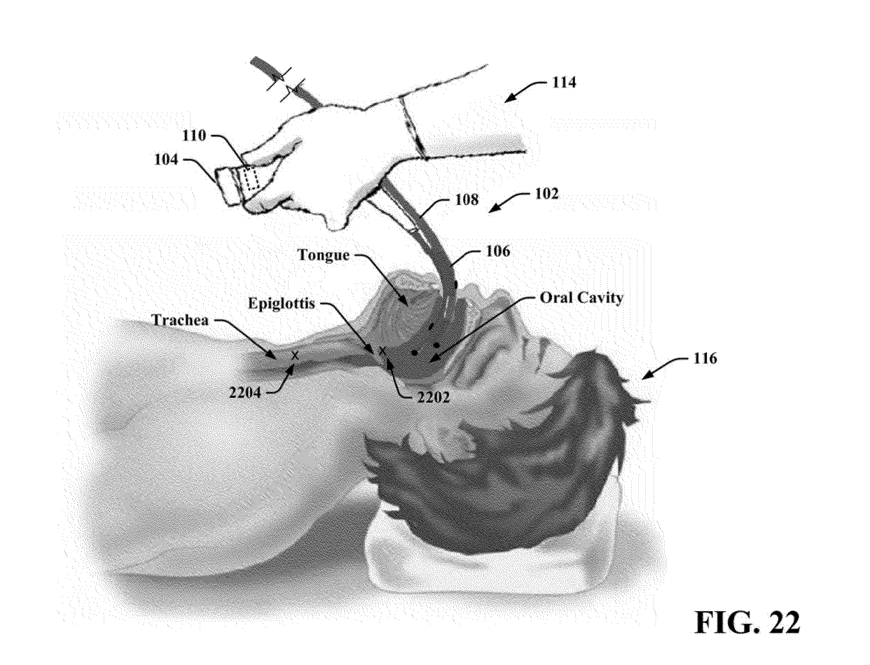

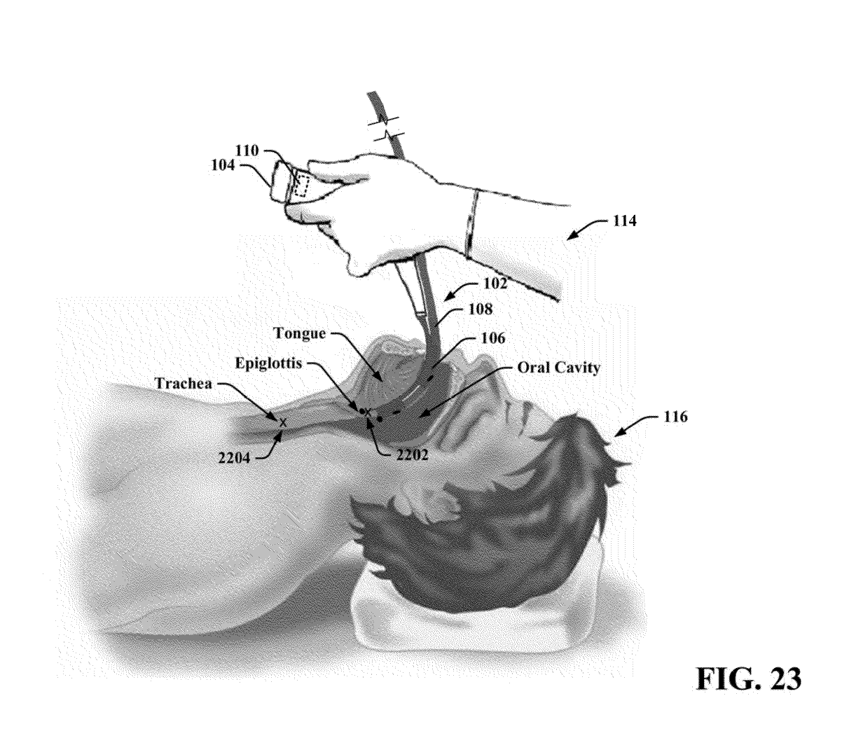

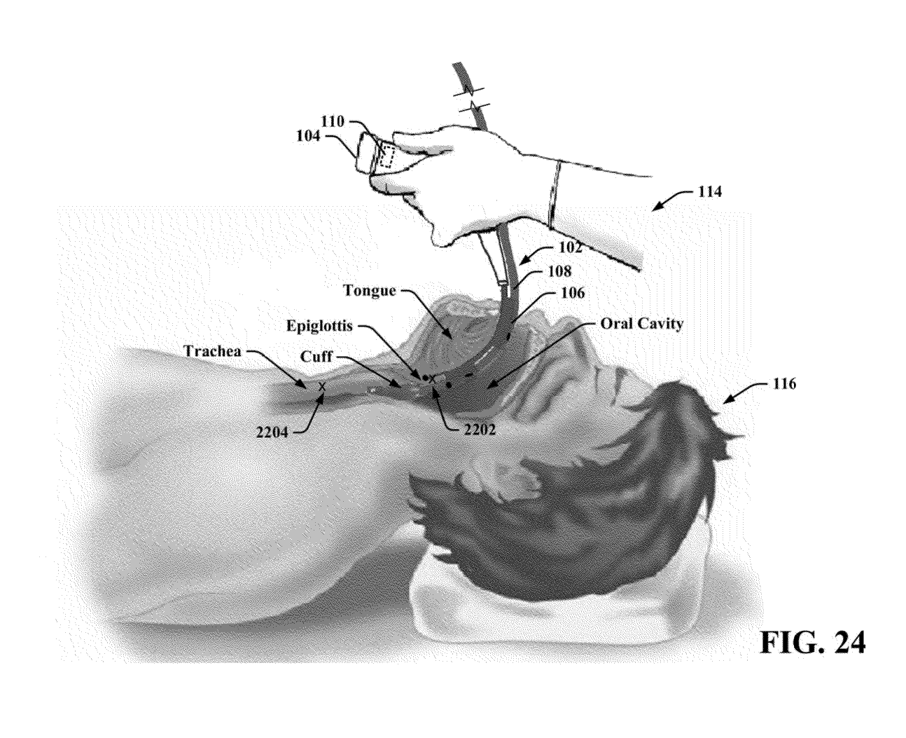

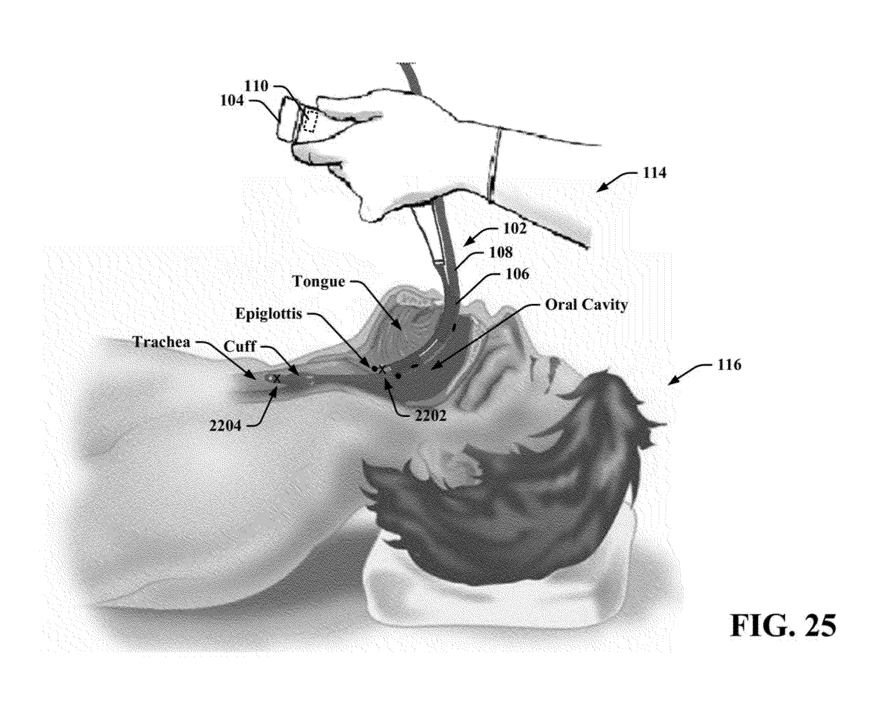

FIGS. 22-25 provide a pictorial demonstration of an example intubation procedure facilitated using airway management apparatus 102 in accordance with various aspects and embodiments described herein. As shown in FIGS. 22-25, two target locations within the patient are identified. A first target location 22012 is located at or near the epiglottic vallecula. A second target location 2204 is located in the middle the trachea. In FIG. 22, the articulating blade 106 is maneuvered into the oral cavity by the operator 114. In FIG. 23, the articulating blade is positioned at the first target location 2202 and maneuvered to pull back the epiglottis and reveal the laryngeal aperture and the vocal cords. In FIG. 24, the endotracheal tube 108 is advanced through the laryngeal aperture past the vocal cords, and in FIG. 25, the distal end of the endotracheal tube 108 reaches the second target location 2204.

Throughout the procedure, feedback regarding the current position and orientation of the articulating blade 106 and/or the endotracheal tube 108 can be provided to the operator (e.g., via analysis component 2102 and guidance component 2104). In addition, the targeting component 2106 can determine, and the guidance component 2104 can provide, feedback regarding how the operator 114 should move and manipulate (e.g., articulate, change length, etc.) the articulating blade 106 and/or the endotracheal tube 108 to guide the articulating blade 106 and/or the endotracheal tube 108 to target locations 2202 and 2204, respectively.

With reference back to FIG. 21, in various embodiments, the control circuit 110 can automatically articulate and maneuver the articulating blade 106 via the blade control component 2002 to correctly and safely depress the patient's tongue and pull back the patient's epiglottis to reveal the patient's laryngeal aperture and vocal cords. For example, the targeting component 2106 can generate and send, to the blade control component 2002, one or more command signals that identify the determined trajectory path for the articulating blade 106 and/or the determined one or more movements that effectuate brining the articulating blade 106 to the target location along the trajectory path. Based on reception of the one or more control signals, the blade control component 2002 can cause the articulating blade 106 to perform the one or more movements. For example, the blade control component 2002 can automatically control the mechanical movement (e.g., via one or more motors 2006) of the airway management apparatus 102 to move the articulating blade forward, backward, upward, downward, left, right, etc. to cause the articulating blade 106 to move along the trajectory path to the target location. In another example, the blade control component 2002 can automatically adjust the curvature of the articulating blade, the length of the articulating blade, the orientation of one or more articulating blade components (e.g., separate blades) at different articulation joints/points, etc.

In some implementations, the control circuit 110 can also automatically control advancement of the endotracheal tube 108 through the oral cavity and the laryngeal aperture between the vocal cords into a target location within the patient's trachea, in a quick (e.g., less than 3 minutes) and efficient manner with little or no physical irritation or trauma to the patient. For example, the targeting component 2106 can generate and send, to the tube control component 2004, one or more command signals that identify the determined trajectory path for the endotracheal tube 108 and/or the determined one or more movements that effectuate brining the endotracheal tube 108 to the target location along the trajectory path. Based on reception of the one or more control signals, the tube control component 2004 can cause the endotracheal tube 108 to perform the one or more movements. For example, the tube control component 2004 can automatically control the mechanical movement (e.g., via one or more motors 2006) of the airway management apparatus to move the endotracheal tube 108 forward, backward, upward, downward, left, right, etc. to cause the endotracheal tube 108 to move along the trajectory path to the target location.

With these automatic or semi-automatic maneuvering capabilities of the airway management apparatus 102, the operator 114 can efficiently and safely perform laryngoscopy to expose the trachea and vocal cords and insert an endotracheal tube 108 into the patient's trachea while operating the airway management apparatus with a single hand. For example, the operator 114 can essentially hold the handle 104 of the airway management apparatus 102 with a single hand in a steady position outside the patient's mouth with the articulating blade 106 positioned over the tongue and within the oral cavity. The operator 114 can then direct or request, via an input device or component of the airway management apparatus (e.g., a physical start/stop button, joystick, etc., a touchscreen GUI, speech input, etc.) the airway management apparatus 102 to automatically perform intubation. In response, the control circuit 110 can react by controlling the movement of the articulating blade 106 and/or the endotracheal tube 108 to effectuate the intubation process. For example, the control circuit 110 can cause the articulating blade 106 to articulate and move within the patient's oral cavity to reveal the laryngeal aperture and drive the endotracheal tube into the patient's trachea. Upon correct positioning of the endotracheal tube 108 within the patient's trachea, the control circuit 110 can cause the articulating blade 106 to automatically release the endotracheal tube 108, and the operator 114 can then carefully pull the airway management apparatus 102 away from the patient's mouth.

In other implementations, the operator 114 can control and manipulate the articulating blade 106 and/or the endotracheal tube 108, and the airway management apparatus 102 can automatically assist and enhance the performance of the operator 114. For example, the airway management apparatus 102 (e.g., via the control circuit 110) can automatically adapt the articulation and configuration of the articulating blade 106 to assist the operator in correctly and safely exposing the laryngeal aperture and depressing the tongue. For example, if the analysis component determines the operator is applying too much or too little pressure with the articulating blade 106 (e.g., based on received presser sensor data), the analysis component 2102 can determine a movement for the articulating blade 106 that corrects pressure application. The analysis component 2102 can then automatically direct (e.g., via an issued command), the blade control component 2002 of the airway management apparatus 102 can to adapt the configuration of the articulating blade using the determined movement to accommodate the correct amount of pressure. In another example, the analysis component 2102 can direct the tube control component 2004 to perform a movement that corrects a location of the endotracheal tube 108 when the operator improperly positions the endotracheal tube 108 past a target location within the trachea. For instance, the analysis component 2102 can determine that the endotracheal tube 108 should be retracted M mm within the trachea. The analysis component 2102 can then automatically retract the endotracheal tube 108 in response to positioning, by the operator, of the endotracheal tube 108 past the target location within the trachea.

In another example, the analysis component 2102 can determine, based on received sensor data (e.g., image data, chemical sensor data, pressure sensor data, etc.) that the operator 114 of the airway management apparatus 102 has successfully positioned the articulating blade, and exposed of the vocal cords and the laryngeal aperture. The analysis component 2102 can then determine is time to advance the endotracheal tube 108 and automatically initiate advancement of the endotracheal tube 108 into the trachea by the tube control component 2004. In yet another example, if the operator fails 114 to hold the articulating blade in a steady position to keep the patient's tongue depressed and the laryngeal aperture exposed (e.g., via a slip of the wrist), the analysis component 2102 can determine how to adjust the configuration (e.g., length, manner of articulation, etc.) and orientation of the articulating blade 106 to correct the operator's mistake and keep the tongue depressed and the laryngeal aperture exposed. The analysis component 2102 can further direct the blade control component 2002 to adjust the configuration and orientation of the articulating blade in the manner determined by the analysis component 2102.

The airway management apparatus 102 can also include a data store within memory 2108 that can retain the data obtained by the one or more sensors 112 and/or evaluated by the analysis component 2102 and targeting component 2106. The data store can be, for example, either volatile memory or nonvolatile memory, or can include both volatile and nonvolatile memory. By way of illustration, and not limitation, nonvolatile memory can include read only memory (ROM), programmable ROM (PROM), electrically programmable ROM (EPROM), electrically erasable programmable ROM (EEPROM), or flash memory. Volatile memory can include random access memory (RAM), which acts as external cache memory. By way of illustration and not limitation, RAM is available in many forms such as static RAM (SRAM), dynamic RAM (DRAM), synchronous DRAM (SDRAM), double data rate SDRAM (DDR SDRAM), enhanced SDRAM (ESDRAM), Synchlink DRAM (SLDRAM), Rambus direct RAM (RDRAM), direct Rambus dynamic RAM (DRDRAM), and Rambus dynamic RAM (RDRAM).

By way of example, the data store can be utilized to document difficult intubations. Thus, data such as images, video, alarms, and the like concerning such intubations can be retained in the data store. Accordingly, the data store can be a flash memory chip that can be removed from the airway management apparatus 102 (e.g., from the handle) and placed in a patient's file. Additionally or alternatively, upon the airway management apparatus 102 being placed in a cradle, data retained in the data store can be archived to hospital records (e.g., upon a server), printed in a report, etc. Further, the data can be archived via a wireless connection to a server associated with the hospital. The data can be archived automatically, periodically, in response to a received request, and so forth. Further, it is contemplated that the data store can similarly be included in any other type of medical device in addition to the airway management apparatus 102 to enable documenting procedures performed upon patients with these other types of medical devices.

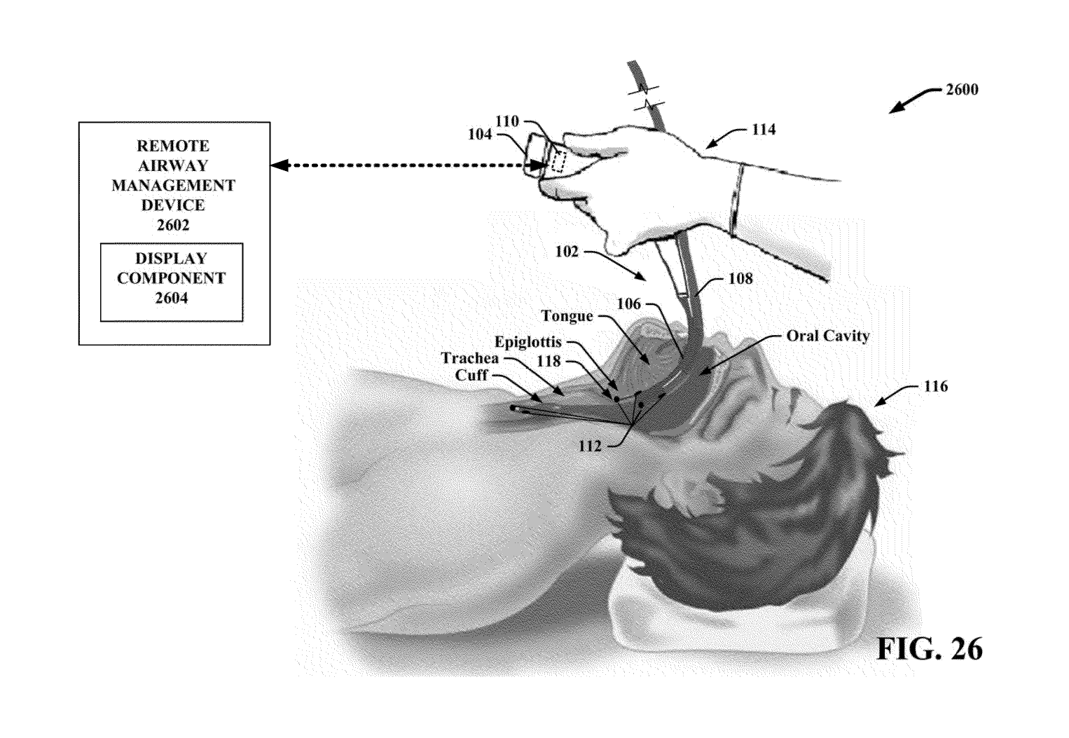

Referring now to FIG. 26, presented is another example system 2600 that facilitates tracheal intubation in accordance with various aspects and embodiments described herein. System 2600 includes same or similar features and functionalities as system 100 with the addition of a remote airway management device 2602. Repetitive description of like elements employed in respective embodiments described herein is omitted for sake of brevity.

In various embodiments, the airway management apparatus 102 is configured to communicate with another device, such as remote airway management device 2602, to exchange information. For example, in some implementations, the airway management apparatus 102 can send information collected via one or more sensors 112 to the remote airway management device 2602 for processing, storage, and/or relaying to yet another device. The sensor data can include image data, chemical analyte concentration data, pressure data, motion data, etc. The remote airway management device 2602 can send the sensor data in real-time or substantially real-time as it is generated to facilitate real-time processing by the remote airway management device 2602.

For example, the airway management apparatus 102 can provide image data (e.g., still images and/or video) to the remote airway management device 2602 captured via one or more cameras located on the articulating blade 106 and/or the endotracheal tube 108. The remote airway management device 2602 can further render the image data via a display component 2604 (e.g., wherein the display component 2604 can include hardware, such as a display screen, and software that facilitates generating and presenting image data). In an aspect, the image data can be sent to the remote airway management device 2602 in real-time or substantially real-time as it is captured. The image data can further be displayed via the display component 2604 in real-time or substantially real-time as it is received, thereby facilitating a live or current view of the oral cavity and associated internal anatomical features during an intubation procedure.

In an aspect, the image data can be associated with depth information and the display component 2604 can generate and display three-dimensional imagery that depicts the articulating blade 106 and/or the endotracheal tube 108 in relation to the anatomical features included in the images. The display component 2604 can also ennable stereoscopic visualization of the laryngeal aperture allowing for depth perception to improve endotracheal tube placement success. For example, the display component 2604 can combine two or more images to create a composite image with depth (e.g., three-dimensional image).

In other implementations, the processing of sensor data provided by analysis component 2102, guidance component 2104 and targeting component 2106 can be performed by the remote airway management device. For example, the remote airway management device 2602 can determine a current location of the articulating blade and/or the endotracheal tube 108 and provide feedback regarding how to move and maneuver the airway management apparatus during an intubation procedure.

In one or more embodiments, the remote airway management can also provide remote control capability of the airway management apparatus 102. In one implementation, the remote airway management device 2602 can allows a remote user (e.g., located in a different room, city, country, etc. from the airway management apparatus 102) to operate and control the airway management apparatus 102 to intubate or facilitate intubating a patient.

For example, the remote airway management device 2602 can allow a user of the remote airway management device 2602 to provide input for application by the blade control component 2002 and the tube control component 2004. The input can identify how to move, maneuver, articulate, advance, etc., the articulating blade 106 and/or the endotracheal tube 108. The remote airway management device 2602 can further generate and send control messages, to the airway management apparatus 102 including control commands based on the input information and instructing the airway management apparatus 102 to execute the control commands. For example, after determining how the articulating blade 106 and/or the endotracheal tube 108 should be moved, articulated, advanced, etc., within the oral cavity, a user of the remote airway management device 2602 can provide input commanding or requesting the airway management apparatus 102 to move, articulate, advance, etc., the articulating blade 106 and/or the endotracheal tube 108 accordingly. Based on reception of the command or request, the blade control component 2002 and the tube control component 2004 can cause the articulating blade 106 and/or the endotracheal tube 108 to move, articulate, advance, etc., as requested. In other embodiments, the manner of movement for the articulating blade 106 and/or the endotracheal tube 108 can be automatically determined by the remote airway management device 2602, and the remote airway management device 2602 can further automatically (e.g., without user input) issue commands to effectuate the movement at the airway management apparatus 102.

Although not depicted, in accordance with various aspects of systems 100 and 2600 associated with automatic control and/or remote control of airway management apparatus 102, in some embodiments, the airway management apparatus 102 is not held by an operator 114 (e.g., a human operator). For example, the airway management apparatus can be mounted on or within a stand, a mount, a bar, or other suitable apparatus configured to hold the airway management apparatus 102 in a steady position above the patient's mouth.

The remote airway management device 2602 can include a wide variety of computing devices. For example, the remote airway management device 2602 can include, but is not limited to, a personal computing (PC) device, such as a smartphone, a tablet PC, a notebook, a personal digital assistant (PDA), a wearable device, or another type of handheld computing device. In one implementation, the remote airway management device 2602 includes a head-mounted device (e.g., glasses, goggles, etc.,) including a display component 2604. In some embodiments, the remote airway management device 2602 includes a PC that is associated with the operator 114. In another example, the remote airway management device 2602 can be any type of device with a monitor or display.

The remote airway management device 2602 can be located within close proximity of the airway management apparatus 102 (e.g., within the operating room) or outside of a local vicinity of the airway management apparatus 102. For instance, the remote airway management device 2602 can enable communicating with the airway management apparatus 102 over an infrastructure based network (e.g., cellular network). Thus, a specially trained individual located anywhere in the world can be presented with feedback from the one or more sensors 112. Further, this individual can control manipulation of the articulating blade 106, the endotracheal tube 108, and/or the data capture via the one or more sensors from the remote location.

The remote airway management device 2602 and the airway management apparatus 102 can be configured to communicate using various wired or wireless communication protocols configured to facilitate communication between devices over various distances. For example, the respective devices can wirelessly communicate with one another using communication protocols including but not limited to: a near field communication (NFC) based protocol, a BLUETOOTH® technology-based protocol (e.g., BLUETOOTH® low energy (BLE) protocol), a ZigBee® protocol, a WirelessHART®, a Z-Wave® based communication protocol, an advanced and adaptive network topology (ANT) based protocol, a radio frequency (RF) based communication protocol, an Internet Protocol (IP) based communication protocol (e.g. hyper text transfer protocol (HTTP), session initiation programming (SIP), IP version 6 over low power wireless personal area networks (6LoWPAN), etc.), a cellular communication protocol, an ultra-wideband (UWB) technology-based protocol, or other forms of communication including both proprietary and non-proprietary communication protocols.

In various embodiments, communication between the remote airway management device 2602 and the airway management apparatus 102 can be facilitated over a personal area network (PAN) or a local area network (LAN) (e.g., a Wireless Fidelity (Wi-Fi) network) that can provide for communication over greater distances than the NFC or BLE protocol and provide other advantages (e.g., stronger encryption protocols). In other embodiments, the remote airway management device 2602 and the airway management apparatus 102 can communicate with one another and/or another device (e.g., another server device or a second external device) over a wide area network (WAN) using HTTP-based communication protocols.

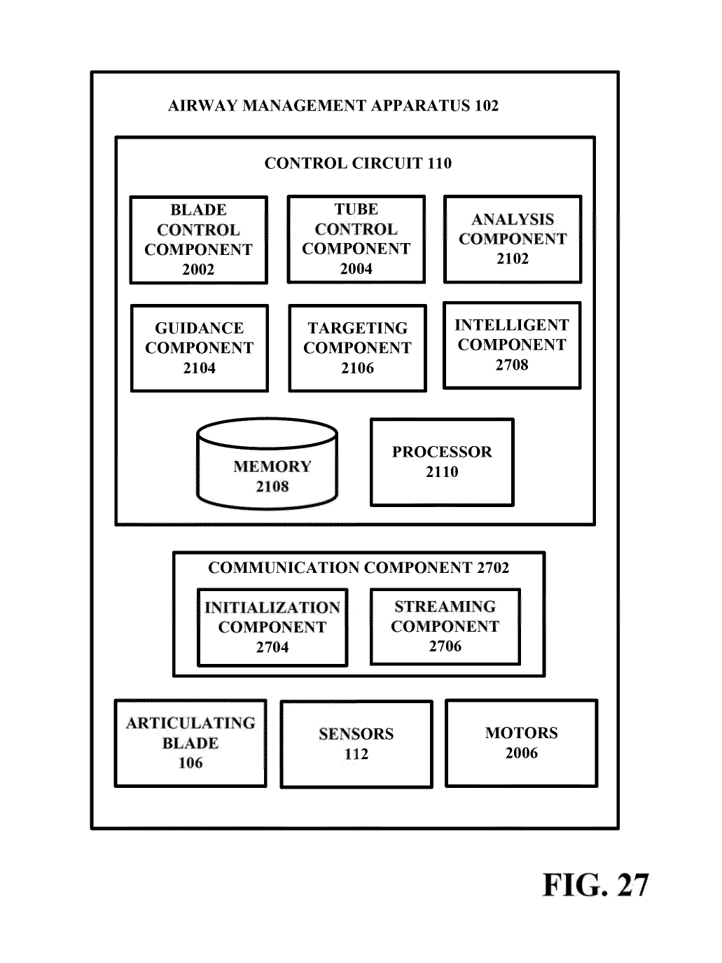

FIG. 27 presents a high-level block diagram of example airway management apparatus 102 in accordance with one or more additional embodiments described herein. Repetitive description of like elements employed in respective embodiments described herein is omitted for sake of brevity.

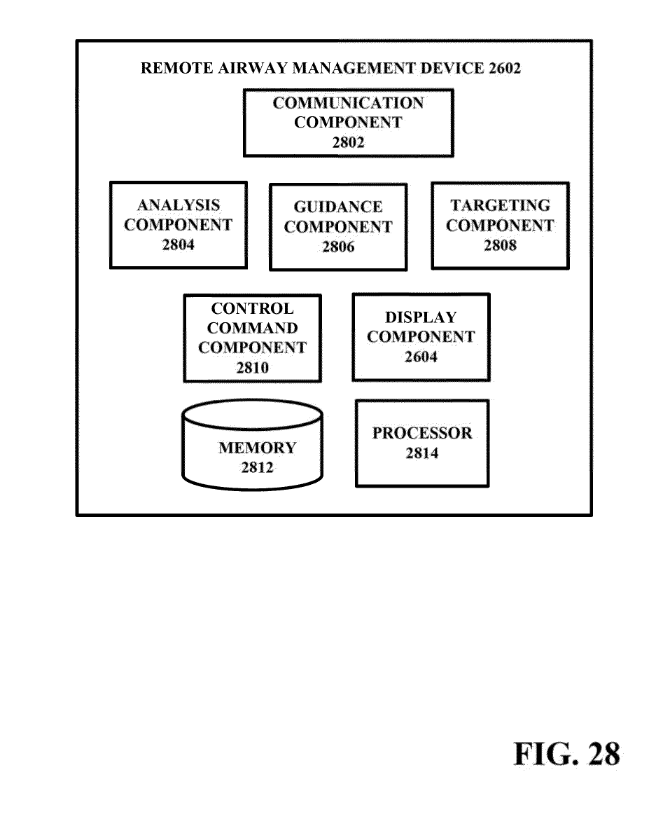

With reference to FIGS. 27 and 28, the airway management apparatus 102 can include communication component 2702 that can enable wired or wireless communication between the remote airway management device 2602 and the airway management apparatus 102. In various exemplary embodiments, the communication component 2702 is configured to facilitate telemetry communication between the remote airway management device 2602 and the airway management apparatus 102. For example, the communication component 2702 can include or be various hardware and software devices associated with establishing and/or conducting a telemetry communication between the remote airway management device 2602 and the airway management apparatus 102. For example, communication component 2702 can control operation of a transmitter-receiver or transceiver (not shown) of the airway management apparatus 102 to establish a telemetry session with the remote airway management device 2602 and to control transmission and reception of signals or data packets between the respective devices.

The communication component 2702 can utilize any type of wireless technology to transfer data (e.g., WiFi, 802.11b, g, n, Bluetooth, . . . ). Thus, the communication component 2702 can enable wireless digital transmission of digital images to allow for remote viewing of airway manipulation, digital recording of procedures, porting images to video equipment in place such as anesthesiology monitoring or portable handle communication devices, and so forth. Moreover, the communication component 2702 can receive feedback from one or more of the external device (e.g., remote airway management device 2602); such feedback that can control manipulation of the articulating blade 106 and/or the endotracheal tube 108 by providing a signal to the blade control component 2002 and/or the tube control component 2004, respectively. Accordingly, this type of feedback can enable performing telemedicine.

The communication component 2702 can further include an initialization component 2704 and a streaming component 2706. Moreover, the communication component 2702 can enable the airway management apparatus 102 to communicate with one or more remote devices (e.g., in addition to remote airway management device 2602). The initialization component 2704 can determine whether any external devices (e.g., remote airway management device 2602) are within range. Thus, a list of identities of these external devices can be populated by the initialization component 2704. Thereafter, one or more of the listed devices can be selected for connecting with the airway management apparatus 102.

By way of illustration, a monitor can be positioned in an operating room in which the airway management apparatus 102 is being employed. The initialization component 2704 can identify that the monitor is within proximity and set up transfer of data to the monitor. For example, the monitor can automatically be initialized by the initialization component 2704; thus, upon moving within range of the monitor, transmission can occur between the communication component 2702 and the monitor to enable display upon the monitor of data collected by the airway management apparatus 102. Additionally or alternatively, the initialization component 2704 can create a list of available devices including the monitor, and a selection may be made based upon a user input, a preference, a ranking, security levels, etc.

The streaming component 2706 can enable real-time transfer of data between the airway management apparatus 102 and the remote airway management device 2602. Thus, the streaming component 2706 can allow for an image obtained with via one or more cameras on the articulating blade 106 and/or the endotracheal tube 108 of a patient's oral cavity to be displayed upon a PDA or any other external interface in real-time as the airway management apparatus 102 is manipulated within the oral cavity. Further, the streaming component 2706 can allow for the data to be transmitted to a disparate device for storage (e.g., a remotely located data store).

The control circuit of airway management apparatus 102 can further include an intelligent component 2708 that can be employed by the airway management apparatus 102. For example, the intelligent component 2708 can infer where the articulating blade and/or endotracheal tube 108 is located relative to one or more anatomical features associated with a patient's oral cavity. In another example, the intelligent component 2708 can infer how to manipulate, articulate, move, etc., the articulating blade 106 and/or the endotracheal tube based on received sensor data. Pursuant to another example, the intelligent component 2708 can infer potential errors in use associated with the airway management apparatus 102 (e.g., misplacement of the endotracheal tube 108) and yield a corresponding alarm.

It is to be understood that the intelligent component 2708 can provide for reasoning about or infer states of the system, environment, and/or user from a set of observations as captured via events and/or data. Inference can be employed to identify a specific context or action, or can generate a probability distribution over states, for example. The inference can be probabilistic—that is, the computation of a probability distribution over states of interest based on a consideration of data and events. Inference can also refer to techniques employed for composing higher-level events from a set of events and/or data. Such inference results in the construction of new events or actions from a set of observed events and/or stored event data, whether or not the events are correlated in close temporal proximity, and whether the events and data come from one or several event and data sources. Various classification (explicitly and/or implicitly trained) schemes and/or systems (e.g., support vector machines, neural networks, expert systems, Bayesian belief networks, fuzzy logic, data fusion engines . . . ) can be employed in connection with performing automatic and/or inferred action in connection with the claimed subject matter.

A classifier is a function that maps an input attribute vector, x=(x1, x2, x3, x4, xn), to a confidence that the input belongs to a class, that is, f(x)=confidence(class). Such classification can employ a probabilistic and/or statistical-based analysis (e.g., factoring into the analysis utilities and costs) to prognose or infer an action that a user desires to be automatically performed. A support vector machine (SVM) is an example of a classifier that can be employed. The SVM operates by finding a hypersurface in the space of possible inputs, which hypersurface attempts to split the triggering criteria from the non-triggering events. Intuitively, this makes the classification correct for testing data that is near, but not identical to training data. Other directed and undirected model classification approaches include, e.g., naïve Bayes, Bayesian networks, decision trees, neural networks, fuzzy logic models, and probabilistic classification models providing different patterns of independence can be employed. Classification as used herein also is inclusive of statistical regression that is utilized to develop models of priority.

FIG. 28 illustrated is a high-level block diagram of example remote airway management device 2602 in accordance with one or more embodiments described herein. Repetitive description of like elements employed in respective embodiments described herein is omitted for sake of brevity.

With reference to FIGS. 26 and 28, remote airway management device 2602 is configured to communicate with airway management apparatus 102 to facilitate various aspect of tracheal intubation using the airway management apparatus. For example, remote airway management device 2602 can receive, in real-time or substantially real-time, image data (e.g., still images and/or video data) captured via one or more cameras (e.g., sensors 112) located on or within the articulating blade 106 and/or the endotracheal tube 108 while located within the oral cavity. The display component 2604 can display the image data during the intubation procedure to provide a clear view of various anatomical features within the oral cavity during intubation (e.g., the laryngeal opening), thereby facilitating performance of the intubation procedure by an operator of the airway management apparatus (e.g., operator 114 and/or a remote operator of the airway management apparatus 102). The display component 2604 can also generate, using depth information associated with the captured imaged data, a three-dimensional view of the anatomical features included in the image data relative to one another and the articulating blade 106 and/or the endotracheal tube 108.

In addition or in the alternative to processing and displaying image the image data captured via one or more cameras located on or within the articulating blade 106 and/or the endotracheal tube 108 during an intubation procedure, the remote airway management apparatus can provide remote processing of sensor data captured via the one or more sensors to dynamically determine where the articulating blade 106 and/or the endotracheal tube is located within the oral cavity relative to one or more anatomical features during the intubation procedure. The remote airway management device can further provide detailed visual and/or audible feedback to the operator of the airway management apparatus identifying the current location of the articulating blade 106 and/or the endotracheal tube relative to the anatomical features. The remote airway management device 2602 can further determine and provide feedback regarding how the articulating blade 106 and/or the endotracheal tube 108 should be articulated, moved and maneuvered throughout the intubation procedure to moved.

Further, in some embodiments the remote airway management device 2602 can enable remote control of the airway management apparatus 102. For example, a user of the remote airway management device 2602 can employ the device to issue and send control commands to the airway management apparatus instructing the airway management apparatus 102 to articulate, move, maneuver, etc., the articulating blade 106 and/or the endotracheal tube 108 in s defined manner. The airway management apparatus 102 can further cause articulating blade 106 and/or the endotracheal tube 108 to perform the movement defined by the control commands in response to reception of the commands (e.g., via blade control component 2002 and/or tube control component 2004). In another implementation, the remote airway management device 2602 can determine and generate the control commands based on a determination regarding how the articulating blade 106 and/or the endotracheal tube 108 should be articulated, moved, maneuvered, etc., (e.g., based on analysis of the received sensor data). According to this implementation, the remote airway management device 2602 can automatically control movement of the articulating blade 106 and/or the endotracheal tube 108 to perform an intubation procedure, thereby eliminating or minimizing human error associated with performance of the intubation procedure.

In order to perform the various features and functionalities described above, in addition to the display component 2604, the remote airway management device 2602 can include communication component 2802, analysis component 2804, guidance component 2806, targeting component 2808, control command component 2810, memory 2812, and processor 2814. One or more of the components of remote airway management device 2602 constitute machine-executable component(s) embodied within machine(s), e.g., embodied in one or more computer readable mediums (or media) associated with one or more machines. Such component(s), when executed by the one or more machines, e.g., computer(s), computing device(s), virtual machine(s), etc. can cause the machine(s) to perform the operations described. The remote airway management device 2602 can include memory 2812 for storing the computer executable components and instructions, and processor 2814 to facilitate operation of the computer executable components and instructions.

Communication component 2802 can include same or similar features and functionality as communication component 2702. For example, communication component 2802 is configured to facilitate wired or wireless communication between remote airway management device 2602 and the airway management apparatus 102. For example, the communication component 2802 can include or be various hardware and software devices associated with establishing and/or conducting a telemetry communication between the remote airway management device 2602 and the airway management apparatus 102. For example, communication component 2802 can control operation of a transmitter-receiver or transceiver (not shown) of the remote airway management device 2602 to establish a telemetry session with the airway management apparatus 102 and to control transmission and reception of signals or data packets between the remote airway management device 2602 and the airway management apparatus 102. The communication component 2802 can facilitate telemetry communication between the remote airway management device 2602 and the airway management apparatus 102 using a variety of telemetry communication protocols.

Analysis component 2804, guidance component 2806, and targeting component 2808 can also include same or similar features and functionalities as analysis component 2102, guidance component 2104, and targeting component 2106. Repetitive description is omitted for sake of brevity. Control command component 2810 is configured to generate and send (e.g., via communication component 2802) control signals including one or more parameters for a manner of movement of the articulating blade 106 and/or the endotracheal tube 108 during an intubation procedure. In some implementations, a user of the remote airway management device 2602 can provide input identifying the desired manner of movement. For example, using a suitable input device/mechanism (e.g., a button, joystick, switch, lever, touch screen, voice command, sensor, mouse, trigger, etc), the user can identify a manner of articulation for the articulating blade 106 and/or a speed of movement of the articulating blade 106 and/or the endotracheal tube 108 forward and backward. Based on the received user input, the control command component 2810 can generate a control command identifying the manner of movement and send the control command to the airway management apparatus 102 for implementation. In other implementations, input identifying a manner of movement for the articulating blade 106 and/or the endotracheal tube 108 is provided automatically via the analysis component 2804 and/or the targeting component 2808.

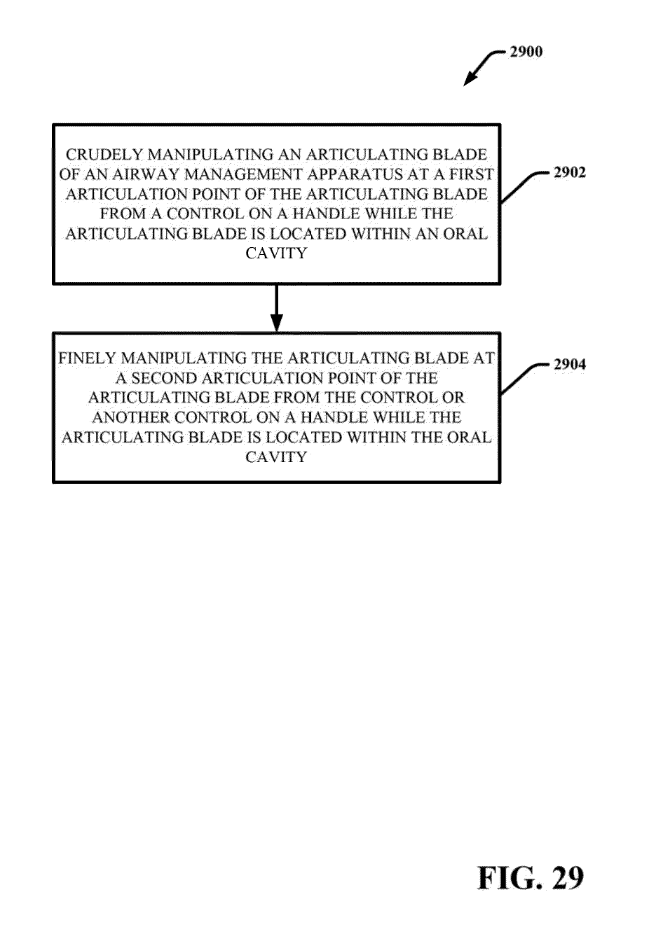

FIGS. 29-34 illustrate methodologies in accordance with the claimed subject matter. For simplicity of explanation, the methodologies are depicted and described as a series of acts. It is to be understood and appreciated that the subject innovation is not limited by the acts illustrated and/or by the order of acts, for example acts can occur in various orders and/or concurrently, and with other acts not presented and described herein. Furthermore, not all illustrated acts may be required to implement the methodologies in accordance with the claimed subject matter. In addition, those skilled in the art will understand and appreciate that the methodologies could alternatively be represented as a series of interrelated states via a state diagram or events.

Referring to FIG. 29, illustrated is an example method 2900 facilitates intubating a patient using an airway management apparatus in accordance with one or more aspects and embodiments described herein. In an exemplary embodiment, the airway management apparatus is airway management apparatus 102 and the like (e.g., airway management apparatus 700). Repetitive description of like elements employed in respective embodiments described herein is omitted for sake of brevity.

At 2902, an articulating blade (e.g., articulating blade 106) of an airway management apparatus (e.g., airway management apparatus 102 and/or 700) is crudely manipulated at a first articulation point (e.g., medium control articulation point 702) of the articulating blade while the articulating blade is located within the oral cavity. For example, the articulating blade can be articulated to position one or more cameras included on or within the articulating blade (e.g., incorporated into the articulating blade, mounted upon the articulating blade, etc.) at the base of the tongue looking upwards towards the vocal cords. In contrast to conventional techniques where manipulation of the articulating blade is conducted while outside of the oral cavity, manipulation of the laryngoscope articulating blade can occur within the oral cavity in connection with the claimed subject matter, thus, repeated removal and reinsertion of the articulating blade can be mitigated. At 2904, the articulating blade is finely manipulated via a second articulation point (fine control articulation point 704) of the articulating blade. The fine articulation, for example, can enable moving a tip of the articulating blade to move the epiglottis and pulling back the epiglottis, thereby yielding a clearer view of the vocal cords and the laryngeal aperture. It is contemplated that the crude and fine manipulation of the articulating blade can be effectuated mechanically, via an electric signal, and so forth.

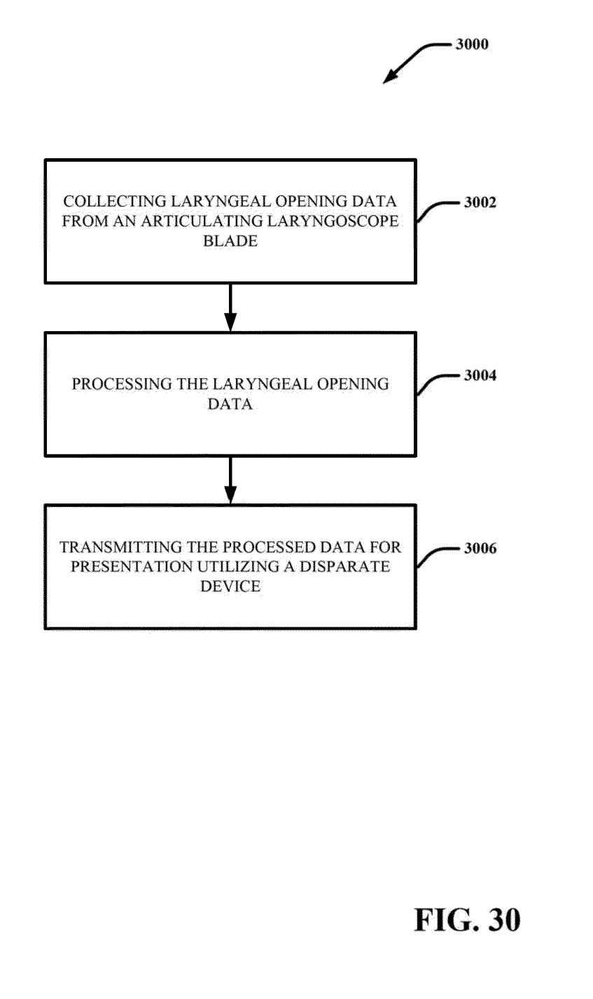

Turning to FIG. 30, illustrated is another example method 3000 facilitates intubating a patient using an airway management apparatus in accordance with one or more aspects and embodiments described herein. In an exemplary embodiment, the airway management apparatus is airway management apparatus 102 and the like (e.g., airway management apparatus 700). Repetitive description of like elements employed in respective embodiments described herein is omitted for sake of brevity.

At 3002, laryngeal opening data can be collected from an articulating laryngoscope articulating blade (e.g., articulating blade 106). For example, data can be obtained utilizing digital cameras mounted upon and/or incorporated into the articulating laryngoscope articulating blade. Further, the articulating blade can be maneuvered to position the cameras with a clear view to the vocal cords. At 3004, the laryngeal opening data can be processed. For instance, data from a plurality of digital cameras can be combined to yield a stereoscopic view of the vocal cords (e.g., via analysis component 2102). At 3006, the processed data can be transmitted (e.g., via communication component 2702) for presentation utilizing a disparate device (e.g., remote airway management device 2602). The data can be transmitted wirelessly, for instance. Moreover, the processed data can be transferred to any type of disparate device that can yield an output. Thus, for example, the processed data can be sent wirelessly to a monitor in an operating room, a cell phone, a PDA, etc. Further, the disparate device can render an output in real time. Accordingly, as the laryngoscope articulating blade is articulated within the oral cavity, a display can be rendered upon the disparate device in real time that shows a view of the vocal cords from the base of the tongue.

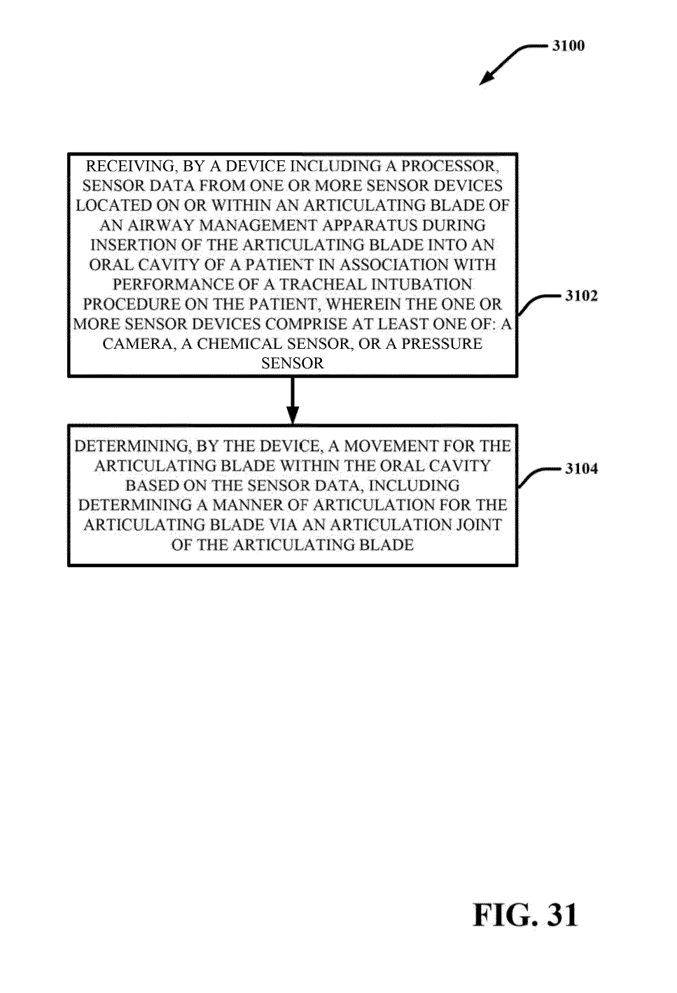

FIG. 31 illustrates another example method 3100 facilitates intubating a patient using an airway management apparatus in accordance with one or more aspects and embodiments described herein. In an exemplary embodiment, the airway management apparatus is airway management apparatus 102 and the like (e.g., airway management apparatus 700). Repetitive description of like elements employed in respective embodiments described herein is omitted for sake of brevity.

At 3102, a device including a processor, (e.g., the handle 104 of airway management apparatus 102 and/or a remote airway management device 2602), receives sensor data from one or more sensor devices located on or within an articulating blade (e.g., articulating blade 106) of an airway management apparatus (e.g., airway management apparatus 102 and/or 700) during insertion of the articulating blade into an oral cavity of a patient in association with performance of a tracheal intubation procedure on the patient. The one or more sensor devices include at least one of: a camera, a chemical sensor, or a pressure sensor. At 3104, the device determines a movement for the articulating blade within the oral cavity based on the sensor data (e.g., via analysis component 2102, targeting component 2106, analysis component 2804 or targeting component 2808). For example, the determining the movement can include determining a manner of articulation for the articulating blade via an articulation joint of the articulating blade.

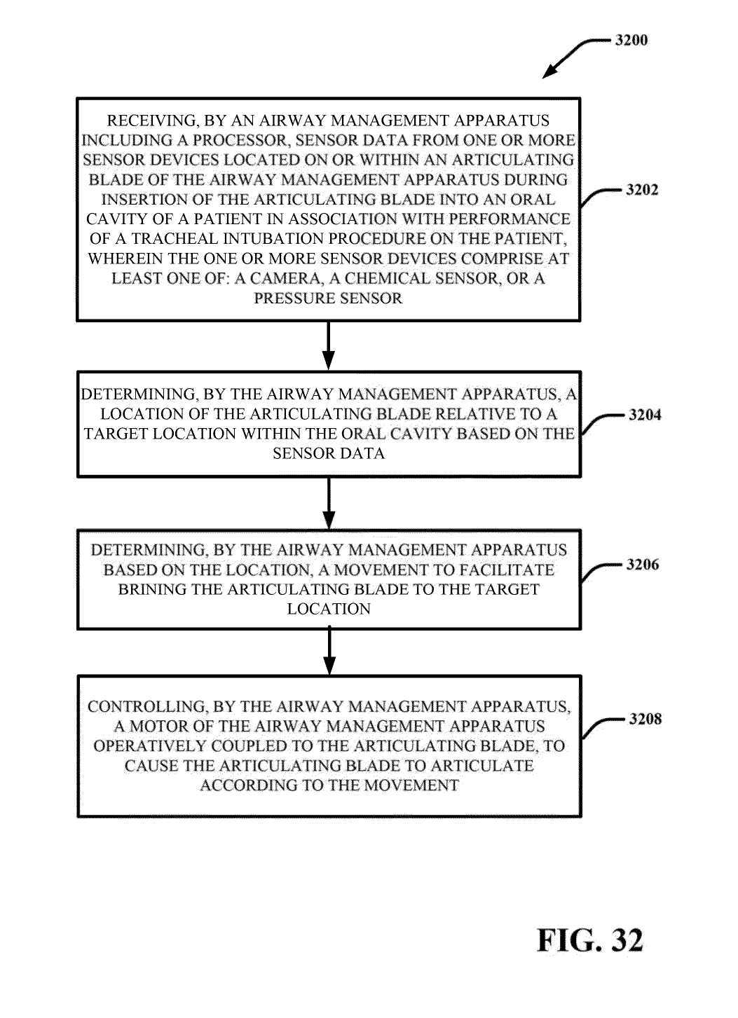

FIG. 32 illustrates another example method 3200 facilitates intubating a patient using an airway management apparatus in accordance with one or more aspects and embodiments described herein. In an exemplary embodiment, the airway management apparatus is airway management apparatus 102 and the like (e.g., airway management apparatus 700). Repetitive description of like elements employed in respective embodiments described herein is omitted for sake of brevity.

At 3102, an airway management apparatus including a processor, (e.g., the handle 104 of airway management apparatus 102 and/or 700), receives sensor data from one or more sensor devices located on or within an articulating blade (e.g., articulating blade 106) of the airway management apparatus (e.g., airway management apparatus 102 and/or 700) during insertion of the articulating blade into an oral cavity of a patient in association with performance of a tracheal intubation procedure on the patient. The one or more sensor devices include at least one of: a camera, a chemical sensor, or a pressure sensor. At 3204, the airway management apparatus determines a location of the articulating blade relative to a target location within the oral cavity based on the sensor data. (e.g., via analysis component 2102). At 3206, the airway management apparatus determines, based on the location, a movement to facilitate brining the articulating blade to the target location. (e.g., via targeting component 2106). At 3208, the airway management apparatus controls a motor of the airway management apparatus operatively coupled to the articulating blade, to cause the articulating blade to articulate according to the movement.

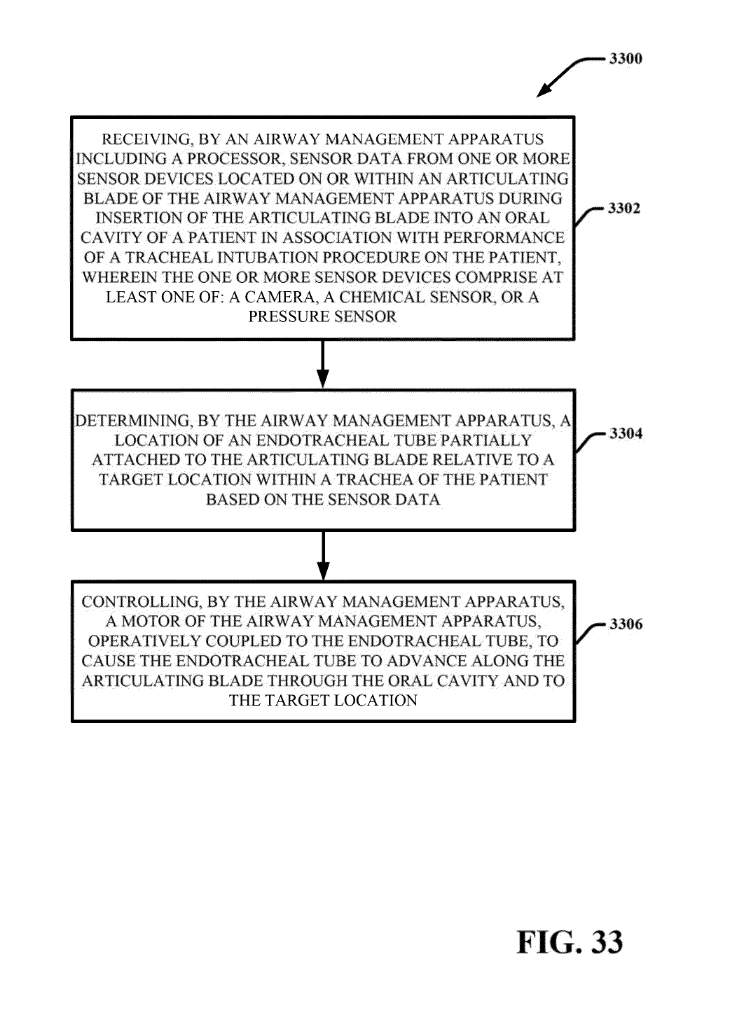

FIG. 33 illustrates another example method 3300 facilitates intubating a patient using an airway management apparatus in accordance with one or more aspects and embodiments described herein. In an exemplary embodiment, the airway management apparatus is airway management apparatus 102 and the like (e.g., airway management apparatus 700). Repetitive description of like elements employed in respective embodiments described herein is omitted for sake of brevity.

At 3302, an airway management apparatus including a processor, (e.g., the handle 104 of airway management apparatus 102 and/or 700), receives sensor data from one or more sensor devices located on or within an articulating blade (e.g., articulating blade 106) of the airway management apparatus (e.g., airway management apparatus 102 and/or 700) during insertion of the articulating blade into an oral cavity of a patient in association with performance of a tracheal intubation procedure on the patient. The one or more sensor devices include at least one of: a camera, a chemical sensor, or a pressure sensor. At 3304, the airway management apparatus determines a location of an endotracheal tube (e.g., endotracheal tube 108) partially attached to the articulating blade relative to a target location (e.g., the second target location 2204) within a trachea of a patient based on the sensor data. At 3306, the airway management apparatus controls a motor of the airway management apparatus, operatively coupled to the endotracheal tube to cause the endotracheal tube to advance along the articulating blade through the oral cavity and to the target location.

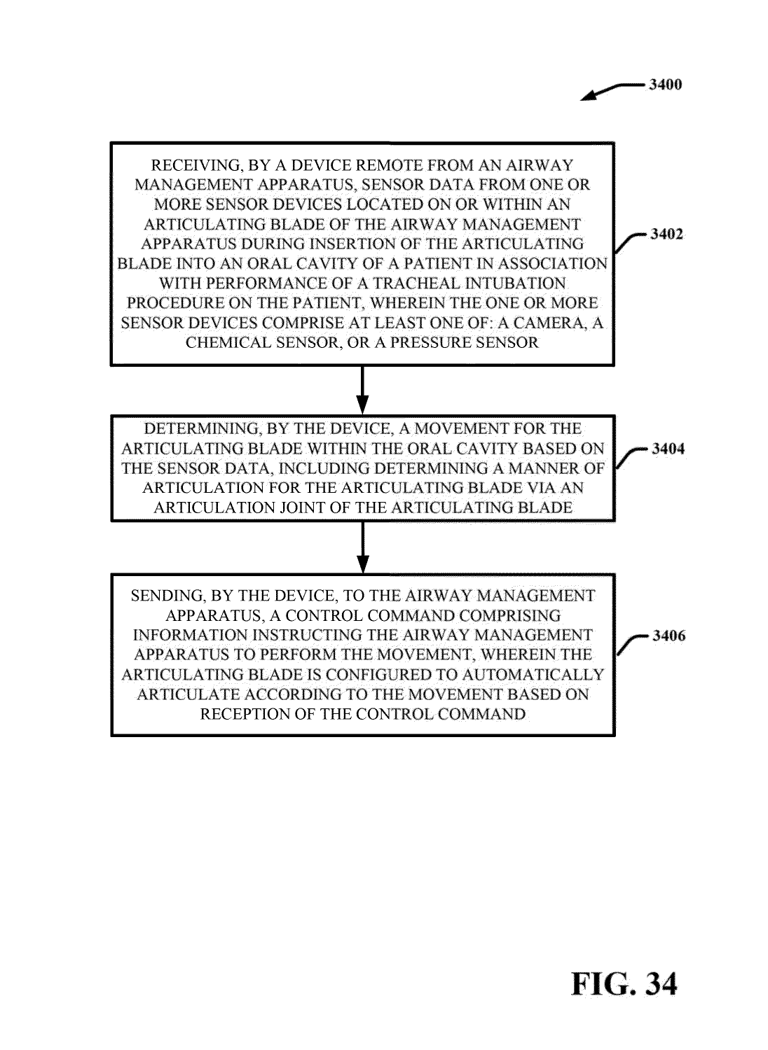

FIG. 34 illustrates another example method 3400 facilitates intubating a patient using an airway management apparatus in accordance with one or more aspects and embodiments described herein. In an exemplary embodiment, the airway management apparatus is airway management apparatus 102 and the like (e.g., airway management apparatus 700). Repetitive description of like elements employed in respective embodiments described herein is omitted for sake of brevity.

At 3402, a device remote from an airway management apparatus (e.g., remote airway management device 2602) receives sensor data from one or more sensor devices located on or within an articulating blade (e.g., articulating blade 106) of an airway management apparatus (e.g., airway management apparatus 102 and/or 700) during insertion of the articulating blade into an oral cavity of a patient in association with performance of a tracheal intubation procedure on the patient. The one or more sensor devices include at least one of: a camera, a chemical sensor, or a pressure sensor. At 3404, the device determines a movement for the articulating blade within the oral cavity based on the sensor data (e.g., via analysis component 2102, targeting component 2106, analysis component 2804 or targeting component 2808). For example, the determining the movement can include determining a manner of articulation for the articulating blade via an articulation joint of the articulating blade. At 3406, the device sends, to the airway management apparatus, a control command including information instructing the airway management apparatus to perform the movement, wherein the articulating blade is configured to automatically articulate according to the movement based on reception of the control command.

In order to provide additional context for implementing various aspects of the claimed subject matter, FIGS. 13-14 and the following discussion is intended to provide a brief, general description of a suitable computing environment in which the various aspects of the subject innovation may be implemented. For instance, FIGS. 13-14 set forth a suitable computing environment that can be employed in connection with generating and/or utilizing replicas of states. While the claimed subject matter has been described above in the general context of computer-executable instructions of a computer program that runs on a local computer and/or remote computer, those skilled in the art will recognize that the subject innovation also may be implemented in combination with other program modules. Generally, program modules include routines, programs, components, data structures, etc., that perform particular tasks and/or implement particular abstract data types.

Moreover, those skilled in the art will appreciate that the inventive methods may be practiced with other computer system configurations, including single-processor or multi-processor computer systems, minicomputers, mainframe computers, as well as personal computers, hand-held computing devices, microprocessor-based and/or programmable consumer electronics, and the like, each of which may operatively communicate with one or more associated devices. The illustrated aspects of the claimed subject matter may also be practiced in distributed computing environments where certain tasks are performed by remote processing devices that are linked through a communications network. However, some, if not all, aspects of the subject innovation may be practiced on stand-alone computers. In a distributed computing environment, program modules may be located in local and/or remote memory storage devices.

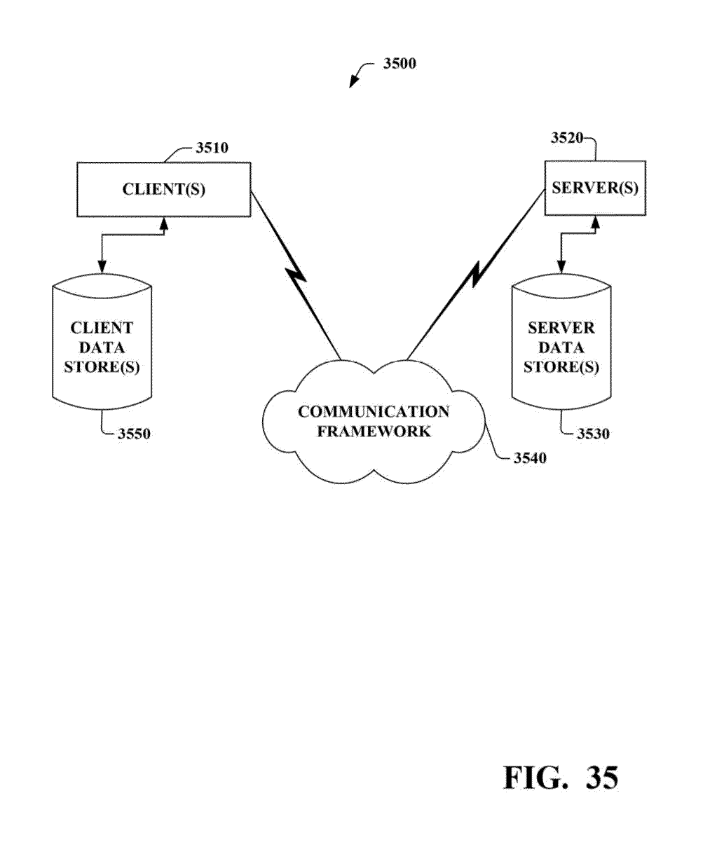

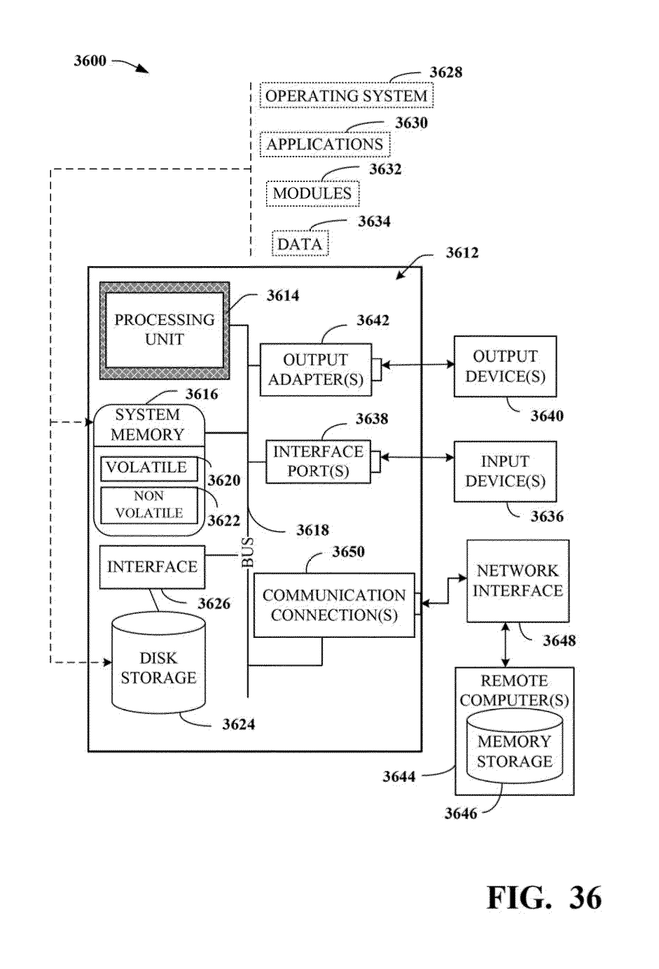

FIG. 35 is a schematic block diagram of a sample-computing environment 3500 with which the claimed subject matter can interact. The system 3500 includes one or more client(s) 3510. The client(s) 3510 can be hardware and/or software (e.g., threads, processes, computing devices). The system 3500 also includes one or more server(s) 3520. The server(s) 3520 can be hardware and/or software (e.g., threads, processes, computing devices). The servers 3520 can house threads to perform transformations by employing the subject innovation, for example.

One possible communication between a client 3510 and a server 3520 can be in the form of a data packet adapted to be transmitted between two or more computer processes. The system 3500 includes a communication framework 3540 that can be employed to facilitate communications between the client(s) 3510 and the server(s) 3520. The client(s) 3510 are operably connected to one or more client data store(s) 3550 that can be employed to store information local to the client(s) 3510. Similarly, the server(s) 3520 are operably connected to one or more server data store(s) 3530 that can be employed to store information local to the servers 3520.

With reference to FIG. 36, an exemplary environment 3600 for implementing various aspects of the claimed subject matter includes a computer 3612. The computer 3612 includes a processing unit 3614, a system memory 3616, and a system bus 3618. The system bus 3618 couples system components including, but not limited to, the system memory 3616 to the processing unit 3614. The processing unit 3614 can be any of various available processors. Dual microprocessors and other multiprocessor architectures also can be employed as the processing unit 3614.

The system bus 3618 can be any of several types of bus structure(s) including the memory bus or memory controller, a peripheral bus or external bus, and/or a local bus using any variety of available bus architectures including, but not limited to, Industrial Standard Architecture (ISA), Micro-Channel Architecture (MSA), Extended ISA (EISA), Intelligent Drive Electronics (IDE), VESA Local Bus (VLB), Peripheral Component Interconnect (PCI), Card Bus, Universal Serial Bus (USB), Advanced Graphics Port (AGP), Personal Computer Memory Card International Association bus (PCMCIA), Firewire (IEEE 1394), and Small Computer Systems Interface (SCSI).

The system memory 3616 includes volatile memory 3620 and nonvolatile memory 3622. The basic input/output system (BIOS), containing the basic routines to transfer information between elements within the computer 3612, such as during start-up, is stored in nonvolatile memory 3622. By way of illustration, and not limitation, nonvolatile memory 3622 can include read only memory (ROM), programmable ROM (PROM), electrically programmable ROM (EPROM), electrically erasable programmable ROM (EEPROM), or flash memory. Volatile memory 3620 includes random access memory (RAM), which acts as external cache memory. By way of illustration and not limitation, RAM is available in many forms such as static RAM (SRAM), dynamic RAM (DRAM), synchronous DRAM (SDRAM), double data rate SDRAM (DDR SDRAM), enhanced SDRAM (ESDRAM), Synchlink DRAM (SLDRAM), Rambus direct RAM (RDRAM), direct Rambus dynamic RAM (DRDRAM), and Rambus dynamic RAM (RDRAM).

Computer 3612 also includes removable/non-removable, volatile/non-volatile computer storage media. FIG. 36 illustrates, for example a disk storage 3624. Disk storage 3624 includes, but is not limited to, devices like a magnetic disk drive, floppy disk drive, tape drive, Jaz drive, Zip drive, LS-100 drive, flash memory card, or memory stick. In addition, disk storage 3624 can include storage media separately or in combination with other storage media including, but not limited to, an optical disk drive such as a compact disk ROM device (CD-ROM), CD recordable drive (CD-R Drive), CD rewritable drive (CD-RW Drive) or a digital versatile disk ROM drive (DVD-ROM). To facilitate connection of the disk storage devices 3624 to the system bus 3618, a removable or non-removable interface is typically used such as interface 3626.

It is to be appreciated that FIG. 36 describes software that acts as an intermediary between users and the basic computer resources described in the suitable operating environment 3600. Such software includes an operating system 3628. Operating system 3628, which can be stored on disk storage 3624, acts to control and allocate resources of the computer system 3612. System applications 3630 take advantage of the management of resources by operating system 3628 through program modules 3632 and program data 3634 stored either in system memory 3616 or on disk storage 3624. It is to be appreciated that the claimed subject matter can be implemented with various operating systems or combinations of operating systems.

A user enters commands or information into the computer 3612 through input device(s) 3636. Input devices 3636 include, but are not limited to, a pointing device such as a mouse, trackball, stylus, touch pad, keyboard, microphone, joystick, game pad, satellite dish, scanner, TV tuner card, digital camera, digital video camera, web camera, and the like. These and other input devices connect to the processing unit 3614 through the system bus 3618 via interface port(s) 3638. Interface port(s) 3638 include, for example, a serial port, a parallel port, a game port, and a universal serial bus (USB). Output device(s) 3640 use some of the same type of ports as input device(s) 3636. Thus, for example, a USB port may be used to provide input to computer 3612, and to output information from computer 3612 to an output device 3640. Output adapter 3642 is provided to illustrate that there are some output devices 3640 like monitors, speakers, and printers, among other output devices 3640, which require special adapters. The output adapters 3642 include, by way of illustration and not limitation, video and sound cards that provide a means of connection between the output device 3640 and the system bus 3618. It should be noted that other devices and/or systems of devices provide both input and output capabilities such as remote computer(s) 3644.

Computer 3612 can operate in a networked environment using logical connections to one or more remote computers, such as remote computer(s) 3644. The remote computer(s) 3644 can be a personal computer, a server, a router, a network PC, a workstation, a microprocessor based appliance, a peer device or other common network node and the like, and typically includes many or all of the elements described relative to computer 3612. For purposes of brevity, only a memory storage device 3646 is illustrated with remote computer(s) 3644. Remote computer(s) 3644 is logically connected to computer 3612 through a network interface 3648 and then physically connected via communication connection 3650. Network interface 3648 encompasses wire and/or wireless communication networks such as local-area networks (LAN) and wide-area networks (WAN). LAN technologies include Fiber Distributed Data Interface (FDDI), Copper Distributed Data Interface (CDDI), Ethernet, Token Ring and the like. WAN technologies include, but are not limited to, point-to-point links, circuit switching networks like Integrated Services Digital Networks (ISDN) and variations thereon, packet switching networks, and Digital Subscriber Lines (DSL).

Communication connection(s) 3650 refers to the hardware/software employed to connect the network interface 3648 to the bus 3618. While communication connection 3650 is shown for illustrative clarity inside computer 3612, it can also be external to computer 3612. The hardware/software necessary for connection to the network interface 3648 includes, for exemplary purposes only, internal and external technologies such as, modems including regular telephone grade modems, cable modems and DSL modems, ISDN adapters, and Ethernet cards.

What has been described above includes examples of the subject innovation. It is, of course, not possible to describe every conceivable combination of components or methodologies for purposes of describing the claimed subject matter, but one of ordinary skill in the art may recognize that many further combinations and permutations of the subject innovation are possible. Accordingly, the claimed subject matter is intended to embrace all such alterations, modifications, and variations that fall within the spirit and scope of the appended claims.

In particular and in regard to the various functions performed by the above described components, devices, circuits, systems and the like, the terms (including a reference to a “means”) used to describe such components are intended to correspond, unless otherwise indicated, to any component which performs the specified function of the described component (e.g., a functional equivalent), even though not structurally equivalent to the disclosed structure, which performs the function in the herein illustrated exemplary aspects of the claimed subject matter. In this regard, it will also be recognized that the innovation includes a system as well as a computer-readable medium having computer-executable instructions for performing the acts and/or events of the various methods of the claimed subject matter.

In addition, while a particular feature of the subject innovation may have been disclosed with respect to only one of several implementations, such feature may be combined with one or more other features of the other implementations as may be desired and advantageous for any given or particular application. Furthermore, to the extent that the terms “includes,” and “including” and variants thereof are used in either the detailed description or the claims, these terms are intended to be inclusive in a manner similar to the term “comprising.”

What is claimed is:

1. An airway management apparatus, comprising:
an articulating blade comprising a plurality of articulation points and a channel configured to receive an endotracheal tube and facilitate passage of the endotracheal tube through the channel;
a handle attached to the articulating blade and comprising a housing;
a motor provided within the housing that is operatively coupled to the articulating blade; and
a blade control component provided on or within the housing, wherein the blade control component is operatively coupled to the motor and controls manipulation of the articulating blade at the plurality of articulation points via the motor.

2. The airway management apparatus of claim 1, further comprising:
a tube control component provided on or within the housing, wherein the tube control component is operatively coupled to the motor or another motor provided within the housing that is configured to operatively couple to the endotracheal tube when the endotracheal tube is provided within the channel, and wherein the tube control component is configured to control passage of the endotracheal tube through the channel via the motor or the other motor.

3. The airway management apparatus of claim 1, wherein the articulating blade is configured to release the endotracheal tube from the channel in response to a release control mechanism associated with the blade control component.

4. The airway management apparatus of claim 1, wherein the articulating blade comprises retainer segments formed on opposing sides of a surface the articulating blade, and wherein the channel comprises a space on the surface of the articulating blade between the retainer segments.

5. The airway management apparatus of claim 4, wherein the retainer segments are configured to hold the endotracheal tube on the surface of the articulating blade when the endotracheal tube is provided within the channel.

6. The airway management apparatus of claim 1, wherein the plurality of articulation points comprise a first articulation point that alters a curvature associated with the articulating blade and a second articulation point that manipulates a tip of the articulating blade.

7. The airway management apparatus of claim 1, further comprising a camera mounted on the articulating blade and configured to capture image data during usage of the airway management apparatus in association with tracheal intubation of a patient.

8. The airway management apparatus of claim 1, further comprising one or more sensor devices located on or within the articulating blade that are configured to capture sensor data during usage of the airway management apparatus in association with performance of an intubation procedure, wherein the one or more sensor devices comprise at least one of: a camera, a chemical sensor, or a pressure sensor.

9. The airway management apparatus of claim 8, wherein the one or more sensor devices comprise an end-tidal carbon dioxide (EtCO₂) sensor configured to measure EtCO₂ levels.

10. The airway management apparatus of claim 8, further comprising:
a transmitter configured to transmit the sensor data to a remote device for processing in association with facilitating the performance of the intubation procedure.

11. The airway management apparatus of claim 10, further comprising:
a receiver configured to receive a control command from the remote device based on the sensor data, the control command identifying a movement for the articulating blade, and wherein the blade control component is configured to cause the articulating blade to perform the movement based on the control command.

12. The airway management apparatus of claim 8, further comprising:
a memory that stores computer executable components; and
a processor that executes at least the following computer executable components stored in the memory:
an analysis component configured to determine a movement for the articulating blade based on the sensor data.

13. The airway management apparatus of claim 12, further comprising:
a guidance component configured to generate an output that informs an operator of the airway management apparatus regarding the movement.

14. The airway management apparatus of claim 12, wherein the analysis component is further configured to direct the blade control component to cause the articulating blade to perform the movement.

15. The airway management apparatus of claim 8, further comprising:
a memory that stores computer executable components; and
a processor that executes at least the following computer executable components stored in the memory:
a targeting component configured to determine, based on the sensor data, a location of the articulating blade relative to a target location within an oral cavity of a patient during the performance of the intubation procedure, and further determine a movement for the articulating blade based on the location.

16. The airway management apparatus of claim 15, further comprising:
a guidance component configured to generate an output that informs an operator of the airway management apparatus regarding the movement.

17. The airway management apparatus of claim 15, wherein the targeting component is further configured to direct the blade control component to cause the articulating blade to perform the movement.

18. An airway management apparatus, comprising:
an articulating blade comprising a plurality of articulation points;
a handle attached to the articulating blade and comprising a housing;
a motor provided within the housing that is operatively coupled to the articulating blade;
a blade control component provided on or within the housing, wherein the blade control component is operatively coupled to the motor and controls manipulation of the articulating blade at the plurality of articulation points via the motor; and
one or more sensor devices located on or within the articulating blade that are configured to capture sensor data during usage of the airway management apparatus in association with performance of an intubation procedure, wherein the one or more sensor devices comprise at least one of: a camera, a chemical sensor, or a pressure sensor.

19. The airway management apparatus of claim 18, further comprising:
a memory that stores computer executable components; and
a processor that executes at least the following computer executable components stored in the memory:
an analysis component configured to determine a movement for the articulating blade based on the sensor data.

20. The airway management apparatus of claim 19, further comprising:
a guidance component configured to generate an output that informs an operator of the airway management apparatus regarding the movement.

21. The airway management apparatus of claim 19, wherein the analysis component is further configured to direct the blade control component to cause the articulating blade to perform the movement.

22. The airway management apparatus of claim 18, further comprising:
a memory that stores computer executable components; and
a processor that executes at least the following computer executable components stored in the memory:
a targeting component configured to determine, based on the sensor data, a location of the articulating blade relative to a target location within an oral cavity of a patient during the performance of the intubation procedure, and further determine a movement for the articulating blade based on the location.

23. The airway management apparatus of claim 22, further comprising:
a guidance component configured to generate an output that informs an operator of the airway management apparatus regarding the movement.

24. The airway management apparatus of claim 22, wherein the targeting component is further configured to direct the blade control component to cause the articulating blade to perform the movement.

25. The airway management apparatus of claim 18, wherein the articulating blade further comprises a channel configured to receive an endotracheal tube and facilitate passage of the endotracheal tube through the channel during the performance of the intubation procedure, the airway management apparatus further comprising:
a tube control component provided on or within the housing that controls passage of the endotracheal tube through the channel via the motor or the other motor.

26. The airway management apparatus of claim 25, further comprising:
a memory that stores computer executable components; and
a processor that executes at least the following computer executable components stored in the memory:
a targeting component configured to determine, based on the sensor data, a location of the endotracheal tube relative to a target location within a patient's trachea during the performance of the intubation procedure on the patient, and further determine a movement for the endotracheal tube based on the location.

27. The airway management apparatus of claim 26, further comprising:
a guidance component configured to generate an output that informs an operator of the airway management apparatus regarding the movement.

28. The airway management apparatus of claim 26, wherein the targeting component is further configured to direct the tube control component to cause the endotracheal tube to perform the movement.

29. The airway management apparatus of claim 26, wherein the sensor data comprises one or more images of internal anatomical features located within the patient's oral cavity and an end-tidal carbon dioxide (EtCO₂) level.

30. A method for facilitating performance of a tracheal intubation procedure using an airway management apparatus, comprising:
receiving, by a device comprising a processor, sensor data from one or more sensor devices located on or within an articulating blade of the airway management apparatus during insertion of the articulating blade into an oral cavity of a patient in association with performance of the tracheal intubation procedure on the patient, wherein the one or more sensor devices comprise at least one of: a camera, a chemical sensor, or a pressure sensor; and
determining, by a the device, a movement for the articulating blade within the oral cavity based on the sensor data, including determining a manner of articulation for the articulating blade via an articulation joint of the articulating blade.

31. The method of claim 30, wherein the device comprises a remote device located remote from the airway management apparatus, the method further comprising:
generating, by the device, an output that informs an operator of the airway management apparatus regarding the movement.

32. The method of claim 30, wherein the device comprises a remote device located remote from the airway management apparatus, the method further comprising:
sending, by the device to the airway management apparatus, a command comprising information instructing the airway management apparatus to perform the movement, wherein the articulating blade is configured to automatically articulate according to the movement based on reception of the command.

33. The method of claim 30, wherein the device comprises a handle of the airway management apparatus that is physically attached to the articulating blade, the method further comprising:
generating, by the device, an output signal that informs an operator of the airway management apparatus regarding the movement.

34. The method of claim 30, wherein the device comprises a handle of the airway management apparatus that is physically attached to the articulating blade, the method further comprising:
controlling, by the device, a motor of the airway management apparatus operatively coupled to the articulating blade, to cause the articulating blade to articulate according to the movement based on the determining.

35. The method of claim 30, further comprising:
determining, by the device, a location of the articulating blade relative to a target location within the oral cavity based on the sensor data, wherein the determining the movement comprises determining the movement based on the location to facilitate brining the articulating blade to the target location.

36. The method of claim 35, wherein the sensor data comprises one or more images of internal anatomical features located within the patient's oral cavity and an end-tidal carbon dioxide (EtCO₂) level.
